(19) United States
(12) Patent Application Publication
(10) Pub. No.: US 2010/0063008 A1
(43) Pub. Date: Mar. 11, 2010

(54) GELATINOUS ELASTOMER COMPOSITIONS

(75) Inventors: Charles J. Matteliano, Kenmore, NY (US); Stephen P. Schaffer, Hamburg, NY (US); Stephen P. Sutton, Troy, NC (US)

Correspondence Address: DARBY & DARBY P.C., P.O. BOX 770, Church Street Station, New York, NY, 10008-0770, US

(73) Assignee: Silipos, Inc., New York, NY (US)

(21) Appl. No.: 12/557,496

(22) Filed: Sep. 10, 2009

Related U.S. Application Data
(60) Provisional application No. 61/095,941, filed on Sep. 10, 2008.
(60) Provisional application No. 61/171,683, filed on Apr. 22, 2009.

(51) Int. Cl. A61K 47/30; A61K 31/60; A61K 31/352; A61K 31/232; A61K 31/045; A61K 31/16; A61P 17/00; A61P 31/00

(52) U.S. Cl. 514/159; 514/772.3; 514/456; 514/552; 514/729; 514/613

(57) ABSTRACT

The present invention is directed to gelatinous elastomer compositions that are useful for topical application of biologically active agents. In certain embodiments, the invention is directed to a gelatinous elastomer composition comprising about 1.0% to 50.0% block copolymer, about 0% to 98% mineral and/or synthetic oil, and about 0.0% to 98% triglyceride oil, about 0-15.0% free fatty acids, about 0-30% of a tack modification agent, about 0-20.0% of a biologically active agent and, optionally a phytosterol, ceramide and/or bisabolol. The gelatinous elastomer compositions are useful for applying a biologically active agent to a mammal. In certain embodiments, the gelatinous elastomer composition is formed into a molded article.

This application claims the benefit of U.S. Provisional Application Nos. 61/095,941 and 61/171,683, filed Sep. 10, 2008, and Apr. 22, 2009, respectively, each of which is incorporated by reference herein in their entireties.

### FIELD OF THE INVENTION

The invention is related to gelatinous elastomer compositions that are useful for topical application of biologically active agents to the human or animal body, as well as methods for topical delivery and treatment based on these compositions.

### BACKGROUND OF THE INVENTION

For decades, thermoplastic elastomer (TPE) block copolymers, having styrenic end groups bound to elastomeric mid-blocks, have been known to form highly plasticized thermo-reversible elastomeric gels when in combination with suitable oils. When oils having sufficient affinity for the mid-block of such polymers, but lesser tendency towards solubilization of polystyrene end-blocks, are used (i.e., mid-block solubilizing oils), solutions are formed from these TPEs at high temperature. In addition, such compositions solidify at or near room/body temperature to yield thermo-reversible gelatinous elastomers. Commonly, mineral oils, and mixtures of mineral oils with other synthetic oils are employed within the composition of such gelatinous materials. The oils that are added to TPE block copolymers are known as plasticizing oils.

Given a sufficiently high percentage of plasticizing oil in TPE gels, together with a suitable styrenic block co-polymer structure (e.g. sufficient molecular weight, proper molecular ratio of styrenic groups to the elastomeric end blocks, etc.), oil gels of this type can exhibit extremely low levels of hardness (down to 20 grams Bloom) and higher levels of hardness up to 3000 grams Bloom. Even at low levels of hardness, related compositions can also display excellent mechanical resilience, high elasticity, and melt processability (e.g. viscous characteristics at temperatures in the range from about 120° C. to 200° C. U.S. Pat. No. 6,552,109 and U.S. Pat. No. 5,334,646 to Chen, and U.S. Pat. No. 5,994,450 to Pearce, which are each incorporated herein by reference in their entireties, also describe such compositions in detail, as well as processing and fabrication of these materials into a broad range of useful articles.

The extreme conformability and softness of these TPE gels often play a crucial role in their application and use. Particularly, styrenic block copolymer oil gels are utilized in many medical cushioning applications wherein they serve to distribute stresses applied to the body.

Beyond applications involving a purely mechanical function, block co-polymer oil gels also find uses stemming from exudation and/or diffusion of oils out of the gel matrix. Specifically, when in contact with many surfaces, including human or animal bodies (as well as other surfaces), such gelatinous compositions are known to exude and or diffuse oil onto the contact surface. Without wishing to be bound to any particular theory, this process may be thought of as a thermodynamic partitioning effect, whereby oil within the gel is exuded due to its affinity for an external material (driving the system in the direction of thermodynamic equilibrium).

It is known to incorporate an additive into a gelatinous material formed into an article for wearing on the body to affect the well-being of the wearer. For example, U.S. Pat. Nos. 5,098,421; 5,167,649; 5,181,914; and 5,330,452, all to Zook and incorporated by reference herein in their entireties, describe various devices comprising a viscoelastic gel pad having pharmacologically active agents incorporated into the gel. U.S. Pat. No. 4,842,931 to Zook, also incorporated by reference herein in its entirety, describes a pad made from a soft viscoelastic gel material containing a high percentage of plasticizing oil for equalizing pressure directed to corns, calluses, bunions and the like. It is also known to apply medication to the skin for the purpose of treating dermal afflictions and for delivering medicine to the body through the dermis. An example of such an externally applied medication is disclosed in U.S. Pat. No. 4,879,274 to Kamiya. which is incorporated by reference herein in its entirety, and describes creams, ointments and the like comprising an α-monoglyceryl ether, a physiologically active material and an oily material. The physiologically active material comprises compounds such as drugs, growth hormones and the like, including vitamins, for example, Vitamins A and B₁₂.

Thus, therapeutic substances can be incorporated within such gels and the gel compositions applied or worn against the skin such that exudation/diffusion of oil (along with any dissolved substances) produces a cosmetic and/or therapeutic benefit, optionally in combination with additional mechanical cushioning benefits. Particularly, it is generally known to incorporate therapeutic agents within TPE gels plasticized using mineral oil, and to employ such compositions as a means to deliver topical treatments. Zook, U.S. Pat. No. 5,167,649, for example, discloses styrenic block copolymer mineral oil gel compositions useful for the topical delivery of pharmacologically active agents dissolved therein, as well as related articles suitable for application to the body. The Zook articles both deliver active compounds via exudation, in addition to providing a cushioning effect for corns, callouses, and other sensitive areas.

Mineral oil based TPE block copolymer gels containing active agents may also include various natural oils, particularly those having therapeutic benefit, which can also be exuded to provide topical delivery of these materials. Gould, U.S. Pat. Nos. 6,117,119 and 6,673,054, which are incorporated by reference in their entireties, contemplates the addition of specific medical grade natural oils (including olive, canola, jojoba, and grapeseed oils), to styrenic block copolymer gels for the purpose of topical delivery. Further, Gould contemplates dissolution of active pharmacological compounds within such gels in a manner similar to that disclosed by Zook (in the patent referenced above).

Though it is known to incorporate pharmacologically active agents within TPE block copolymer oil gels for the purpose of topical delivery, and incorporate natural oils into these compositions, there remains a need for improved oil gel compositions wherein the rate at which biologically active substances are delivered to the body may be controlled and sustained over a broad and useful range. In addition, there remains a need for oil gel compositions wherein processability can be manipulated such that the gels may be practically melt fabricated into a number of useful articles (e.g. via melt coating onto fabrics, melt molding into forms such as pads, and the like). Finally, there remains a need for the simultaneous achievement of useful delivery rates within gel compositions which are practically melt processable.

To this end, the present inventors have discovered improved gelatinous compositions for delivery of triglyceride oils and other biologically active agents to and/or through the skin. The compositions may be used, for example, to deliver biologically active agents such as, for example, skin care agents and/or other therapeutic agents for non-dermal conditions, and also cosmetic agents. The compositions form a cross-linked three-dimensional elastomer network and may thus be formed into articles that may be, for example, applied directly to the skin, or body or internal body cavity or hair of a mammal.

### BRIEF SUMMARY OF THE INVENTION

The present invention is directed to gelatinous elastomer compositions that are useful for topical application of biologically active agents for cosmetic and/or therapeutic treatments.

In certain embodiments, the invention provides a gelatinous elastomer composition comprising about 1.0% to 50.0% block copolymer, about 0% to 98% of a mid-block solubilizing oil, such as a mineral and/or synthetic oil, and about 0.0% to 98% triglyceride oil, and optionally, about 0% to 15.0% free fatty acids, about 0% to 30% of a tack modification agent, about 0% to 20.0% of a biologically active agent and, optionally a phytosterol, ceramide and/or bisabolol.

In other embodiments, the invention provides a method of delivering a triglyceride oil and, optionally, one or more additional biologically active agents to a mammal, comprising contacting said mammal with the gelatinous elastomer composition of the present invention.

In certain embodiments, the invention provides a molded article comprising the gelatinous elastomer composition of the present invention.

In one embodiment, the present invention provides gelatinous elastomer compositions, comprising a block copolymer, and both a mid-block solubilizing oil, such as a mineral and/or synthetic oil, and a triglyceride oil. These gelatinous elastomer compositions have controlled rates of oil exudation for topical delivery applications.

In another embodiment, the present invention provides controlled delivery rate gel compositions having molten viscosities appropriate to enable practical melt processing.

In yet another embodiment, the present invention includes methods of providing cosmetic and medical therapy to humans and animals via the application of the present compositions to the skin, or body or internal body cavity or hair of a mammal.

In yet another embodiment, the present invention provides articles, comprising the novel gel compositions, suitable for application to, or wearing upon, the human and or animal body.

In yet another embodiment is a method for reducing the discoloration and thickness of keloid and hypertrophic scars, comprising the steps of:

a) providing a substrate and a gelatinous elastomer composition of the present invention, the gelatinous elastomer composition comprising one or more of a coconut oil, capric triglycerides, caprylic triglycerides, free fatty acids, high linoleic acid natural oils (for example safflower oil), high oleic acid natural oils, grape seed oil, avocado oil, jojoba oil, canola oil, ceramides, bisabolol, hexyldecanol, Cetylhydroxyproline Palmitamide, Stearic Acid and Brassica Campestris (Rapeseed) Sterols, Padina Pavonica Thallus Extract, aloe, p-menthane 3,8 diol and mixtures thereof;

b) optionally incorporating a therapeutically active agent selected from the group consisting of Vitamins A, B₁₂, C, D, E, and mixtures thereof into a gelatinous elastomer composition of the present invention,

c) bonding the gelatinous elastomer composition to the substrate;

d) forming the substrate having the gelatinous elastomer composition bonded thereto into a body protection article; and

e) wearing the body protection article on the keloid or hypertrophic scar for an extended period of time.

In yet another embodiment are methods for using the present compositions, methods of controlling the rate of oil exudation and/or diffusion from the present compositions, methods of controlling the viscosity of the present compositions, and methods of testing the present compositions in order to discover optimal oil exudation and/or diffusion profiles, and melt processing characteristics.

In yet another embodiment is a method for providing a gelatinous elastomer composition having a desired rate of biologically active agent delivery to the skin or body or internal body cavity or hair of a mammal.

the method comprising:

a) providing a gelatinous elastomer composition of the present invention which comprises a biologically active agent, said composition having a ratio of a triglyceride oil to a mid-block solubilizing oil;

b) contacting the gelatinous elastomer composition with a material capable of absorbing the biologically active agent;

c) measuring the rate at which the biologically active agent is absorbed onto the material;

d) correlating the absorption rate with the delivery rate to the human or animal body;

e) providing an additional gelatinous elastomer composition which comprises a biologically active agent, wherein

if the absorption rate at which the biologically active agent present in the gelatinous elastomer composition of step (a) is lower than the desired delivery rate, the ratio of the triglyceride oil to the mid-block solubilizing oil is increased and

if the absorption rate at which the biologically active agent present in the gelatinous elastomer composition of step (a) is higher than the desired delivery rate, the ratio of the triglyceride oil to the mid-block solubilizing oil is decreased;

f) repeating steps (b)-(d) with the additional gelatinous elastomer composition;

g) providing yet an additional gelatinous elastomer composition of the present invention which comprise a biologically active agent, wherein

if the absorption rate at which the biologically active agent present in the gelatinous elastomer composition of the prior additional gelatinous elastomer composition is lower than the desired delivery rate, the ratio of the triglyceride oil to the mid-block solubilizing oil is increased and

if the absorption rate at which the biologically active agent present in the gelatinous elastomer composition of the prior additional gelatinous elastomer composition is higher than the desired delivery rate, the ratio of the triglyceride oil to the mid-block solubilizing oil is decreased;

h) repeating steps (f) and (g) as many times as are necessary until a gelatinous elastomer composition is provided having the desired rate of delivery.

### BRIEF DESCRIPTION OF THE DRAWINGS

FIG. 1 is an elevational view of a glove according to the present invention.

FIG. 2 is an elevational view of a sock according to the present invention.

FIG. 3 is a graph showing the oil exudation rates of the gel formulations described in Example 5.

### DETAILED DESCRIPTION OF THE INVENTION

The present inventors have unexpectedly discovered significant instability associated with many TPE block co-polymer gel compositions when they contain triglyceride oils. Contrary to teachings in the prior art, natural oils of high polarity (i.e., significantly more polar than those used in typical elastomeric TPE mid-blocks) do not significantly swell or form gels with styrenic block co-polymer TPEs. The present inventors have found that natural oils containing triglycerides (i.e., a triglyceride oil) fail to form stable elastomeric gels with such TPEs, unless the amount of triglyceride oil and other components are carefully controlled. In addition, when triglyceride oils are incorporated into a gel, there is a greater potential for syneresis (i.e., spontaneous weeping or expellation of the plasticizing oil mixture) from the gel. This leads to an unstable gel that is unacceptable for topical delivery.

It is an important goal to provide TPE gels are stable, easily processable, and exude an oil, or an oil blend, and optionally one or more additional active agents, at a rate that is acceptable for topical delivery. To this end, the present inventors have discovered that a significant fraction of a lower polarity mid-block solubilizing oil, e.g., isoparaffin, (i.e., oils which swell/dissolve the mid-block but do not dissolve the polystyrene end groups) is often required in addition to the triglyceride oil. Accordingly, the present inventors have unexpectedly discovered that, when triglyceride oils are combined with other mid-block solubilizing oils in particular ratios, controlled rates of oil delivery (and any biologically active component therein) are achieved which are sufficient to enable applications surrounding delivery of agents to the body. The rates of delivery may be tailored to the application of interest by changing this ratio within the acceptable range.

The present inventors have also discovered unexpected changes in melt processability (e.g. molten viscosity) when triglyceride oils are incorporated into gel compositions. Without wishing to be bound to any particular theory, the relatively polar nature of, e.g., polystyrene end groups are responsible for self assembly and gelation of these materials, such that incorporation of significantly polar oils (including triglyceride oils) improves melt solvation of the polymer at high temperature. The present inventors have discovered a significant lowering of gel melt viscosity when triglyceride oils are substituted for other mid-block solubilizing oils (e.g., mineral oils and isoparaffins) within, e.g., styrenic TPE gels (when all other factors are held constant). Since a lower viscosity is not ideal for melt processing operations (particularly melt coating of gel onto fabrics without unwanted wet through), improved formulations are needed wherein the viscosity may be adjusted and controlled within desirable limits. The present inventors have overcome these limitations through the discovery of TPE gels containing triglyceride oils with other mid-block solubilizing oils in specific ratios. When the inventive combination of oils is used, an increase in melt viscosity is observed which renders triglyceride oil containing TPE gels suitable for processing. Additionally, by modifying the ratio of triglyceride oil to mid-block compatible oil, gel melt viscosity may be raised or lowered depending of the specific application of interest.

In work directed toward related technical solutions, the present inventors have discovered improved gelatinous compositions (comprising both mid-block solubilizing oils and triglyceride oils in specific ratios) for delivery of biologically active agents at controlled rates to and/or into the body. The compositions may be used, for example, to deliver biologically active agents such as, for example, skin care agents and/or other therapeutic agents for non-dermal conditions, and also cosmetic agents. Further, these compositions possess durable soft elastic properties (at body temperatures) enabling the making of articles suitable for wearing on the body of the user, while being suitably thermoplastic and melt processable.

In some embodiments, the compositions of the present invention comprise styrenic block copolymers, having polystyrene end groups and elastomeric mid-blocks, blended with suitable mixtures of mid-block compatible and triglyceride oils in a ratio that provides a useful rate of oil exudation. While the triglyceride oil and or mid-block solubilizing oils, in themselves, may have biological activity, and function within these inventive compositions without addition of other biologically active components, inventive compositions further encompass formulations wherein additional biologically active materials (e.g. active cosmetic and therapeutic substances) may be optionally incorporated within the gel composition such that they are delivered, along with the exuded oil, upon contact with the body.

As stated above, the present inventors have unexpectedly discovered that the ratio of triglyceride oil to mid-block solubilizing oil in the block co-polymer TPE gel has a significant effect upon both the rate of oil exudation and the melt viscosity of the gel. Specifically, they have unexpectedly discovered that the rate of oil exudation is a function of the weight/weight ratio of triglyceride oil to mid-block solubilizing oil, and that this rate increases as this ratio increases. In some embodiments, when the triglyceride oil to mid-block solubilizing oil ratio becomes too high, syneresis of oil takes place. Thus, the ratio of these two oils is an important factor for providing useful gels and articles that are stable and suitable for the present cosmetic and medical applications.

Further, the present inventors have unexpectedly discovered that increasing the triglyceride content of these gel compositions, while holding other factors (e.g., the polymer makeup) constant, lowers melt viscosity such that, through suitable arrangement of polymer type and composition, melt viscosity may be adjusted over a very broad range. Thus, the inventive compositions enable precise adjustment of gel exudation rates (e.g. the delivery rate of active components dissolved therein) over heretofore unachieved/unrecognized useful ranges while, simultaneously, enabling adjustment of melt processability (as a means to facilitate the fabrication of useful articles for delivery of substances to the body).

Compositions

The present invention is directed to compositions that form controlled, stable release triglyceride oil-polymer gels. The compositions comprise one or more block copolymers and one or more triglyceride oil in amounts that, preferably, form low rigidity, non-oriented gelatinous elastomer gels. Such compositions may comprise, for example, a triblock copolymer, e.g., copolymers comprising blocks of styrene-ethylene/butylene-styrene, styrene-ethylene/propylene-styrene, hydrogenated styrene-isoprene/butadiene, hydrogenated styrene-isoprene, hydrogenated styrene-ethylene/butylene-styrene copolymer, and one or more triglyceride oils, and preferably, one or more mid-block solubilizing oils, such as mineral oils, synthetic oil, isoparrafin oils, and ester oils. The compositions are useful for topical application of biologically active cosmetic or therapeutic agents to the skin, body tissues, or hair.

In some embodiments, the compositions described herein comprise a liquid portion, e.g., a triglyceride oil, or a triglyceride oil and one or more additional types of oil (e.g., a mid-block solubilizing oil such as a mineral or synthetic oil), and a thermoplastic elastomer solid fraction. The oil or oils swells the polymer, and together the oil and polymer portions form a cross-linked three dimensional gelatinous elastomer gel network. The oil or oils remain migratory in the composition and the rate of migration can be controlled with formulation and processing, e.g., by changing the identity of the block co-polymer or by changing the ratio the triglyceride oil to mid-block solubilizing oil. The oil portion of the compositions can carry biologically active agents, e.g., emollients, vitamins, humectants, or pharmaceutically or cosmetically active agents.

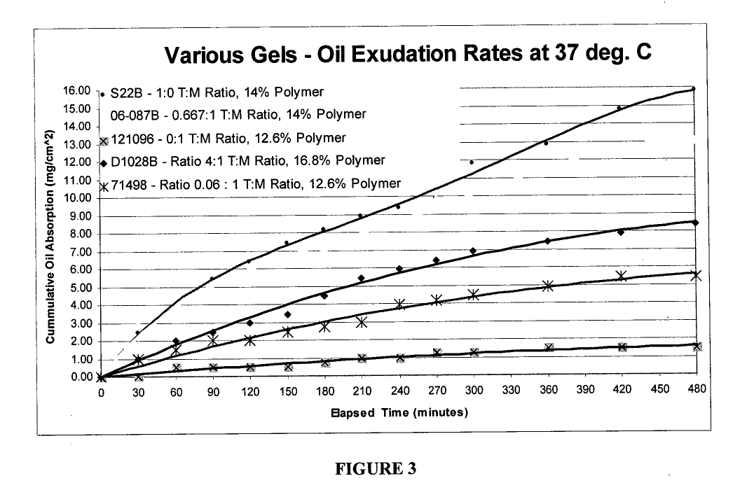

Without wishing to be bound by any particular theory, it is believed that the copolymer end blocks, e.g., styrenic end blocks, are self assembled into nanoscale semi-crystalline polymeric agglomerates (so called “physical,” or thermo-reversible, crosslinks, held together via intermolecular interaction without covalent bonding). Within this structure, the oil mixture, essentially, solubilizes the polymer mid-blocks, while polymer chain ends remain substantially linked by self assembled polystyrene end segments. This phenomenon is illustrated, from example, in U.S. Pat. No. 5,994,450 (See FIG. 3 which shows a very basic conceptual schematic of the likely molecular network/arrangement within this type of gel structure).

In a preferred embodiment, the compositions of the present invention comprise one or more block copolymer, one or more mid-block solubilizing oils, and one or more triglyceride oils, in amounts that that form low rigidity, non-oriented gelatinous elastomer gels which display levels of oil exudation useful for applications wherein biologically active substances are delivered to the human or animal body.

The thermoplastic, heat formable and heat reversible gelatinous elastomer compositions described herein preferably enhance the stability of biologically active agents contained therein and deliver them at a higher rate, compared to compositions known in art. Compositions disclosed herein, for example, exude oil and hence biologically active agents at a rate that is substantially greater than the exudation rate obtained from leading mineral oil formulations that are currently available. The gelatinous elastomer compositions described herein have additional advantageous properties, compared, for example, to foams, pastes and creams (which are sometimes mis-labeled as “gels”). The gelatinous elastomer compositions disclosed herein are mostly oil, which is non-compressible. In some embodiments, the polymer oil gel is thus capable of dissipating pressure and shear forces in a “hydraulic” manner, bounce back and retain shape, and is thus superior to conventional materials, such as foams, pastes and creams, which cannot duplicate this property. In other embodiments, the gels dissipate pressure and shear forces in an elastomeric manner. The gelatinous elastomer compositions also exhibit the desirable properties in that they may be adhered to various fabrics and substrates and molded to various shapes, e.g., by inclusion of a tack modifier agent, can be formulated to be self-adhesive or not adhesive, can be washed and re-used and can slowly release oils, emollients or with the oils can release other biologically active agents to the skin. The rate of dissipation of the oils and therefore biologically active agents can be controlled through formulation chemistry. The gel acts as a reservoir and preserves biologically active agents, thus it can be used to slowly deliver biologically active agents to the skin over long periods of time. Moreover, the gel compositions described herein do not support the growth of bacteria (i.e., they are self anti-microbial, are completely hydro-phobic, and are dermatologically safe).

All concentrations or amounts disclosed herein are expressed as percentage by weight, i.e., w/w.

Gelatinous elastomer compositions generally comprise 1.0% to 50.0% of Block Copolymer, 0% to 98% of Mineral or Synthetic Oil, 0.0% to 98% of Triglyceride Oil, 0.0% to 20.0% of biologically active agent 0% to 15.0% Free Fatty Acids. Gelatinous elastomer compositions may further comprise phytosterols, ceramides and/or bisabolol. Compositions may further comprise 0% to 30% of one or more tack modification agent. Preferred tack modification agents are chosen from the group consisting of hydrogenated synthetic esters, non-hydrogenated synthetic esters, wood rosin esters and other rosins. Block copolymers are generally included at concentrations of 1-50% (w/w), preferably at 4%-25% and more preferably at 10%-25%. Preferably, the block copolymer is a styrenic TPE block copolymer. In one embodiment, a gelatinous elastomer comprises 100 pbw hydrogenated SI/B block copolymers with viscosities of 20-35, 25-150, 60-150, 200-400, and 90 cPs and higher, corresponding to 20 wt % viscosity of 80000 cPs and higher, and 300 to 1600 pbw of a selected plasticizer to achieve 20 to 3000 g bloom with or without an additional copolymer, such as, SBS, SB, SIS, SI, SEP, SEPS, SEBS, SEB, SEP, SEB, PS, PB, EP, EB, PP, PE, and being linear, radial, star, balanced or multiarm. In other preferred embodiments, a gelatinous elastomer comprises 9% to 30% of a blend of Hydrogenated Styrene Isoprene/Butadiene block Copolymer, Hydrogenated Styrene Isoprene block Copolymer or Hydrogenated Styrene-Ethylene/Butylene-Styrene. Other suitable block copolymer suitable for use in the present invention are described in U.S. Pat. No. 7,290,367 to Chen.

Within this broad category of styrenic TPE block copolymers, fully hydrogenated polymers are preferred owing to their general stability and resistance to degradation/oxidation (both during processing and in storage/use). Thus, SEBS and SEPS polymers are generally preferred for use in the inventive gel compositions, with SEBS polymers being especially preferred. Such polymers are particularly exemplified by polymers sold under the tradename KRATON® G manufactured by Kraton Polymers, LLC of Houston Tex., as well as materials sold under the tradename SEPTON® by Kurrary America Inc., Septon B.U., of Pasade, TX.

These styrenic block co-polymers may be employed within inventive gel compositions, either in the form of a single species/grade, or in mixtures of different species in order to manipulate polydispersity. Nonetheless, use of any single molecular weight of such polymers, and or any mixture of such polymers, are within the bounds of inventive gel formulations.

In some embodiments, styrenic TPE polymers described above may be incorporated within inventive gel compositions in amounts ranging from 5% by weight to 45% by weight. Preferably, overall weight percentage of polymer is in the range from 7.0% to 38%. Most preferably, overall weight percentage of polymer is in the range from 8% to 35%.

Plasticizing oils include, for example and without limitation, white mineral oils, triglyceride oils, and synthetically derived oils.

In some embodiments, the present compositions also includes a mid-block solubilizing oil. The term “mid-block solubilizing oil,” as used herein, refers to any liquid which swells/dissolves the elastomeric mid-block of any block co-polymer described above, but which does not dissolve associated end blocks. Such compounds, in general, are capable of forming gels with a given block co-polymer TPE (in isolation and without the addition of other oils/substances).

Among suitable mid-block compatible oils, preferred are those which are of high purity, and sold as suitable for medical/food applications (particularly those manufactured according to USP and or NF standards), and those which are synthetic (e.g. which do not comprise petroleum derived mineral oils since fossil oils are considered by some as non-renewable and or disfavorable from the standpoint of potential impurities).

Mid-block solubilizing oils for use in the present invention are well known in the art. They include, but are not limited to, rubber processing oils such as paraffinic and naphthionic petroleum oils, highly refined aromatic-free paraffinic and naphthionic food and technical grade white petroleum mineral oils, and synthetic liquid oligomers of polybutene, polypropene, polyterpene, etc. The synthetic series process oils are typically high viscosity oligomers which are permanently fluid liquid nonolefins, isoparaffins or paraffins of moderate to high molecular weight. Examples of representative commercially available plasticizing oils include Amoco™ polybutenes, hydrogenated polybutenes and polybutenes with epoxide functionality at one end of the polybutene polymer and ARCO Prime, Duraprime and Tufflo oils. Other white mineral oils include: Bayol, Bernol, American, Blandol, Drakeol, Ervol, Gloria, Kaydol, Litetek, Lyondell's Duraprime series, Marcol, Parol, Peneteck, Primol, Protol, Sonrex, and the like. Generally, plasticizing oils with average molecular weights less than about 200 and greater than about 700 may also be used.

Mineral and/or synthetic oils are present in an amount up to 98%. Preferred amounts of mineral and/or synthetic oils are 1-99%, 10-90%, 20-50%, 30-50% and 25-50%. Specific examples of mineral and synthetic oils are USP-FCC White Mineral Oil such as Duoprime-70, Duoprime 200 or Clarion-70 and also or a Synthetic Hydrogenated Polydecene such as Exxon Mobil Pure-Syn-2, and also or a Synthesized Polyisobutene or Hydrogenated Didecene or Polydecene such as Lipo Products Panalane or Silkflo series, and also or Mineral Oil Substitutes such as Tridecyl Stearate (and) Neopentyl Glycol Dicaprylate/Dicaprate (and) Tridecyl Trimellitate Tridecyl Stearate (and) Tridecyl Trimellitate (and) Dipentaerythrityl Hexacaprylate/Hexacaprate, and also or Dipentaerythrityl Hexacaprylate/Hexacaprate (and) Tridecyl Trimellitate (and) Tridecyl Stearate (and) Neopentyl Glycol Dicaprylate/Dicaprate, such as the Lipovol MOS™ series and also or and synthetic oils or octyl palmitate or other palmitic acids of similar molecular weights and viscosities in the range of 50 to 10000 cPs.

The term “triglyceride oil,” as used herein, refers to any natural or synthetic oil which contains a triglyceride molecule.

Many oils comprising triglyceride molecules are of natural origin, and comprise a mixture of triglyceride species, varying greatly with respect to the structure and nature of bound carboxylic acids. Also, such oils may comprise various impurities, such as waxes, proteins, free carboxylic acids, free alcohols, and a number of other compounds. Nonetheless, all such compositions, whether natural or synthetic in origin, shall be defined as triglyceride oils herein. Preferably, the triglyceride oils of the present invention comprise greater than 50% by weight of triglyceride molecules (as defined above). In addition, all materials meeting this chemical definition, whether liquid or solid at room temperature, whether natural or synthetic in origin, shall be defined herein as a triglyceride oil, and are considered suitable for use in inventive gel compositions (for example, and without limitation, a material such as 76 degree coconut oil, which crystallizes to a solid just above typical room temperature, and which typically comprises a minor fraction of free fatty acids and other impurities, will be defined herein as a triglyceride oil). Additionally, most natural fats contain a complex mixture of individual triglycerides. Because of this, they melt over a broad range of temperatures. Cocoa butter is unusual in that it is composed of only a few triglycerides, one of which contains palmitic, oleic and stearic acids in that order. Triglyceride oil is present in an amount up to 99% (w/w), e.g., 1-99%. Preferred amounts of triglyceride oil are 10%-90%, 20%-80%, 20%-50%, 30%-50%, 25-50%, and 1-10%. Examples of triglyceride oils include, without limitation, Capric Triglyceride, Caprylic Triglyceride, Hydrogenated Vegetable Oil, A Persea Gratissima (Avocado) Oil, Prunus Amygdalys Dulcis (Sweet Almond) Oil, Vitis Vinifera (Grape Seed) Oil, Glycine Soja (Soybean) Oil, Simmonsia Chinensis (Jojoba) Seed Oil, Prunus Armeniaca (Apricot Kernel) Oil, Clear Simmonsia Chinensis (Jojoba) Seed Oil (which is a mono-ester Fatty Acid-Fatty Alcohol), Sesamum Indicum (Sesame) Oil, Carthamus Tinctorius (Hybrid Safflower) Oil, Carthamus Tinctorius (Safflower) Oil, Juglans Regia (Walnut) Oil, Trictum Vulgare (Wheat Germ) Oil, Hellanthus Annuus (Sunflower Seed) Oil, Fractionated Coconut Oil, Guineenis (Palm) Oil, Olea Europaea (Olive) Oil, (Pale Pressed) Ricinus Communis (Castor) Oil, Macadamia Ternifolia Nut Oil, Hydrogenated Soybean Oil, Canola Oil, Rosa Canina Fruit Oil, Lite Rosa Canina Fruit Oil, Corylus Americana (Hazelnut) Oil, Oryza Sativa (Rice Bran) Oil, Balsam Copaiba, Brassica Campestris (Rapeseed) Oil, Rubus Idaeus (Raspberry) Seed Oil, Oleic/Palmitoleic/Linoleic Glycerides, Hydrogenated Avocado Oil, Andiloba Oil, Aloe Vera Oil, Corn Oil, Wheat Oil, Palm Kernel Oil, Brazil Nut Oil, Peanut Oil, Refined Sunflower Seed Oil and other Hydrogenated or non-hydrogenated processed and refined Vegetable, Fruit Seed and Plant Oils and fractionated derivatives thereof.

In addition to commonly known triglycerides, certain oils containing monoglycerides and/or diglycerides have shown promise as plasticizing oils and as partial solubilizing agents for specific bioactive ingredients. Thus, in some embodiments, the eleastomeric gels of the present invention may comprise monoglycerides and/or diglycerides. For purposes of definition, a monoglyceride, more correctly known as a monoacylglycerol, is a glyceride consisting of one fatty acid chain covalently bonded to a glycerol molecule through an ester linkage. Monoacylglycerol can be broadly divided into two groups; 1-monoacylglycerols and 2-monoacylglycerols, depending on the position of the ester bond on the glycerol moiety. Monoacylglycerols can be formed by both industrial chemical and biological processes. They are formed biochemically via release of a fatty acid from diacylglycerol by diacylglycerol lipase or hormone sensitive lipase. Monoacylglycerols are broken down by monoacylglycerol lipase. A diglyceride, or a diacylglycerol (DAG), is a glyceride consisting of two fatty acid chains covalently bonded to a glycerol molecule through ester linkages. One example, shown on the right, is 1-palmitoyl-2-oleoyl-glycerol, which contains side-chains derived from palmitic acid and oleic acid. Diacylglycerols can also have many different combinations of fatty acids attached at both the C-1 and C-2 positions.

Mono- and Diglycerides are commonly added to commercial food products in small quantities. They act as emulsifiers, helping to mix ingredients such as oil and water that would not otherwise blend well. Among the monoglyceride and diglyceride oils suitable for use within inventive gel compositions, most preferred are those which are of high purity, and sold as suitable for medical/food applications (particularly those manufactured according to USP and or NF standards).

In some embodiments, an oil containing one or more of a monoglyceride, diglyceride or triglyceride is incorporated in a specific weight percentage based on the weight of the total oil mixture (the amount of monoglycerides, diglycerides or triglycerides present in the overall mixture of triglyceride oil and mid-block solubilizing oil, and optionally any other oil based additives described herein), in amounts up to slightly above that which will produce syneresis at 20° C. Preferably, the monoglyceride, diglyceride or triglyceride to total oil percentage is adjusted to an amount sufficient to initiate a desired level of oil exudation. Most preferably, the monoglyceride, diglyceride or triglyceride to total oil ratio is optimized, via any of the testing methodologies disclosed herein, to an amount sufficient to provide a desired rate of biologically active substance delivery to the body.

In certain embodiments, the monoglyceride, diglyceride or triglyceride weight percent based on the weight of the total oil mixture is preferred to be at or near the threshold at which spontaneous syneresis of oil from the gel will occur at or near body temperature. Nonetheless, the exact syneresis threshold is difficult to define precisely, and in any case, some inventive embodiments for delivery of components to the body are considered useful very near, or slightly in excess, of the syneresis boundary in a given gel system. Thus, in some preferred embodiments, the percentages of monoglyceride, diglyceride or triglyceride content within the range of 3% to 60% by weight based on the weight of the overall oil mixture within the present gel compositions. Preferable ranges for the percentage of triglyceride content within the overall oil mixture are 5% to 55%, 7% to 51%, and 10% to 50% by weight.

The present inventors have discovered that, in some embodiments of the present invention, the compositions diffuse and or exude oil contained therein, into and/or onto a contacting surface, at rates which are a strong function of the ratio of triglyceride oil weight to the total oil weight within the gel, or as a function of the ratio of the triglyceride oil to the mid-block solubilizing oil (e.g., mineral or synthetic oil). In some embodiments, the addition of triglyceride oils within gel compositions of the present invention enable achievement of exudation rates unachievable in comparable prior art isoparaffin gels (e.g. gels typically comprising mineral oils and having comparable concentrations of polymer at similar levels of mechanical softness, toughness, etc.). The exudation/diffusion rates increase with increased triglyceride oil percentage, with an upper stability limit at which the gel exhibits spontaneous oil syneresis (e.g. spontaneous weeping of oil from the gel surface). Syneresis of oil from within a gel composition generally represents instability of the gel, and leads to separation of the gel into two phases. Thus, in some embodiments, the triglyceride oil is present in a weight percent that is at or below the point at which syneresis occurs. Compositions containing triglyceride oil in an amount at or near the syneresis point may be useful where delivery requires minor phase separation, e.g., when the gels is employed as an ultrasound coupling device.

In certain preferred embodiments, gelatinous elastomer compositions comprise triglyceride oils and mid-block solubilizing oils, e.g., mineral or synthetic oils in a concentration ratio of between about 2:100 to 35:40 or between about 15:60 to 35:40, and more preferably, about 1, 2, 3, 4, 5, 6, or 7 to 50, 60, 70, 80, 90 or 100, or about 28, 29, 30, 31, 32, 33, 34, or 35 to 45, 46, 47, 48, 49, 50, 51, or 52. Further preferred are gelatinous elastomer compositions comprising triglyceride to mid-block solubilizing oils, e.g., mineral or synthetic oils in the aforementioned ratios and from about 8 to 20 parts copolymer. Such compositions provide a gel with the desirable properties of providing the rigidity and elastic properties for easy manufacture into gel coated articles or laminated sheets, withstanding crystallization of the triglyceride portion of formulations, and exhibiting controlled, slow release of oils and/or biologically active agents from the gel matrix. The combination of triglyceride oils and mid-block solubilizing oils, e.g., mineral or synthetic oils is superior to formulations comprising only mineral or synthetic oils, which are typically difficult to formulate such that they provide sufficient exudation of oil deliver effective concentrations of biologically active agents while maintaining gel strength and integrity.

In a preferred embodiment, the triglyceride oil to mid-block solubilizing oil ratio is from about 1:100 to about 3:1, preferably about 33:67 to about 67:33, and more preferably from about 60:40 to about 40:60. In some embodiments the ratio is ratio is about 50:50. In ratios of 0 parts mid-block solubilizing oil to 100 parts triglyceride oil (e.g., coconut oil), it has been shown that instability, crystallization and weak physical properties ensue.

In an alternative embodiment, syneresis and/or weeping is desirable, such as when the gels is employed as an ultrasound coupling device. In some embodiments, syneresis and/or weeping in observed when the triglyceride oil to mid-block solubilizing oils ratio exceeds about 3:1. Thus, the present invention also contemplates gels wherein the triglyceride oil to mid-block solubilizing oil ratio is at or near the ratio at which syneresis and/or weeping begin to occur, preferably no more than 10% above the ratio at which syneresis and/or weeping begin to occur, and more preferably no more than 5% above the ratio at which syneresis and/or weeping begin to occur, and even more preferably no more than 2% above the ratio at which syneresis and/or weeping begin to occur. A determination of the ratio at which syneresis and/or weeping occurs can be determined by one of ordinary skill in the art using known techniques and techniques described in detail herein.

The present inventors have further recognized that a vast number of substances may be dissolved within triglyceride oil/mid-block solubilizing oil mixtures (even to a limited extent), and therefore may be incorporated into gel compositions of the present invention for the purpose of, e.g., delivery to the human or animal body (especially those substances having bioactive and or therapeutic benefit). In such a system, any additive dissolved within the oil comprising a gel will, to some extent, be carried along during oil exudation that occurs when the composition is brought into contact with a surface, e.g., in contact with the skin.

It should be appreciated that the rate of delivery for each a substance may not necessarily be calculated from the oil exudation rate (on the assumption of a concentration of the substance within exudate equivalent to that within the oil fraction of the gel). Particularly, the possibility exists for complex partitioning effects between gel and tissue, respectively; and the partitioning effect will be based on the physical properties of each specific substance, as well as the nature of the other components within the composition. As a particular example, a substance having great affinity for the polymer mid-blocks, similar to that of the mid-block solubilizing oil component, might be expected to be present at a lower percentage, as delivered across a boundary with a more polar substance, than in the oil comprising the gel composition. In such case, the rate of exudation for such a substance is likely to be non-linear as a function of its concentration within the gel.

Nonetheless, the addition of substances to inventive gel compositions, through the mechanism of overall oil/active diffusion/exudation, has been demonstrated by the present inventors, and enables delivery of substances to external surfaces at rates which may be measured, and adjusted, through optimization of the overall oil exudation rates (e.g. via manipulation of the triglyceride/mid-block solubilizing oil ratio and/or the nature of the copolymer). Accordingly, by following the methods of the present invention, gel systems appropriate for controlled delivery of these substances to a surface, e.g., the skin, can be developed and optimization based on the desired use. Oils may be delivered with or without the addition of tack modification agents. Tack modification agents are preferably present in the range of 0%-20% (wt/wt). Tack modification agents include, without limitation, hydrogenated synthetic esters, non-hydrogenated synthetic esters, wood rosin esters and other. Examples of rosins include, for example and without limitation, Eastman Foral, Regalite, Regalrez, Eastotac and Foralyn series resins, Prime Materials Sukorez resins, and other hydrogenated highly processed pine and tree resins and synthetic derivatives thereof.

In some embodiments, given the need for inventive gels to function essentially as a reservoir for the delivery of such biologically active substances to the body, and that they be suitable for storage over reasonable periods of time without unwanted chemical changes to constituents (particularly biologically active substances incorporated therein), the addition of various stabilizing agents is preferred. Additionally, incorporation of various stabilizing agents is preferred to prevent degradation of gel constituents during processing operations (particularly those at elevated temperature). Within the current inventive framework, such stabilizing components may include any substance which may inhibit unwanted chemical changes in components of the gel. Examples of such stabilizing substances include, without limitation, UV absorbers and antioxidants (including BHA and BHT), chelating agents, and other compounds designed to eliminate the presence of undesirable reactive species. Thus, in some preferred embodiments, the inventive gel compositions may comprise such stabilizing substances in percentages ranging up to about 10% by weight.

In some embodiments, it is further desirable for inventive gels to be coupled with agents which limit the rate of growth of micro-organisms both during storage, and in application. In particular, it is preferred that these inventive gels comprise chemical compounds which render them static with respect to the count of bacteria, fungus, mold, and other micro-organisms which may be present either within the body of the gel, or upon the gel surface. Thus, in these embodiments, it is preferred to incorporate various preservative compounds within inventive gel compositions, including, for example and without limitation, any of the paraben compounds, as well as other agents having similar synergistic effect such as glyceryl laurate. Here, it is important to recognize that the effective functioning of such substances may be synergistically enhanced by the presence of the types of stabilizing substances, particularly antioxidants and chelating agents described above. Thus, in these embodiments, it is preferred to incorporate such preservative compounds within inventive gel compositions, and most preferred to incorporate such preservatives alongside some combination of stabilizing substances. In certain preferred embodiments, the inventive gel compositions may comprise such preservatives (not including any synergistic stabilizing compounds) in percentages ranging up to 5%.

Since, in some applications, it may be desirable to modify to the smell, appearance, density, color, hand (e.g. feel upon contact with the body), and or general mechanical characteristics of a given inventive gel, inventive compositions may further comprise substances for such purposes. Examples of such materials include, without limitation, synthetic, inorganic and organic, plant and animal derived fragrances, as well as powders of solid substances such as glass, hollow glass beads, solid glass beads, polymer, cellulose, etc., as well as substances such as esters, waxes, pigments, etc.

In certain embodiments, gelatinous elastomer compositions comprise, consist of or consist essentially of block copolymers and triglyceride oil. Thus, in certain embodiments, gelatinous elastomer compositions comprise, consist of or consist essentially of about 1-50% block copolymer and up to 99% triglyceride oil, about 1-50% block copolymer and 1-99% triglyceride oil, about 1-50% block copolymer and 10-90% triglyceride oil, about 1-50% block copolymer and 20-80% triglyceride oil, about 1-50% block copolymer and 20-50% triglyceride oil, about 1-50% block copolymer and 25-50% triglyceride oil, about 1-50% block copolymer and 30-50% triglyceride oil, about 4-25% block copolymer and up to 99% triglyceride oil, about 4-25% block copolymer and 1-99% triglyceride oil, about 4-25% block copolymer and 10-90% triglyceride oil, about 4-25% block copolymer and 20-80% triglyceride oil, about 4-25% block copolymer and 20-50% triglyceride oil, about 4-25% block copolymer and 25-50% triglyceride oil, about 4-25% block copolymer and 30-50% triglyceride oil, about 10-20% block copolymer and up to 99% triglyceride oil, about 10-20% block copolymer and 1-99% triglyceride oil, about 10-20% block copolymer and 10-90% triglyceride oil, about 10-20% block copolymer and 20-80% triglyceride oil, about 10-20% block copolymer and 20-50% triglyceride oil, about 10-20% block copolymer and 25-50% triglyceride oil, or about 10-20% block copolymer and 30-50% triglyceride oil.

In certain preferred embodiments, gelatinous elastomer compositions comprise, consist of or consist essentially of block copolymer, triglyceride oil and a mid-block solubilizing oil, e.g., a mineral and/or synthetic oil. Thus, in certain embodiments, gelatinous elastomer compositions comprise, consist of or consist essentially of block copolymer, triglyceride oil and a mid-block solubilizing oil, e.g., mineral or synthetic oil, wherein the a combination of block copolymer and triglyceride oil as set forth above combined with a an amount of mineral and/or synthetic oil in a range selected from about up to 98% (w/w), 1-99%, 10-90%, 20-50%, 30-50% and 25-50%.

In other embodiments, gelatinous elastomer compositions comprise 1.0% to 50.0% of Block Copolymer, 50% to 98% of mid-block solubilizing oil, e.g., a mineral or synthetic Oil, 0.0% to 98% of triglyceride Oil, 0.0% to 20.0% of biologically active agent 0% to 15.0% Free Fatty Acids. In a preferred embodiment, a gelatinous elastomer comprises 9% to 30% of a blend of Hydrogenated Styrene Isoprene/Butadiene block Copolymer, Hydrogenated Styrene Isoprene block Copolymer or Hydrogenated Styrene-Ethylene/Butylene-Styrene, 0% to 70% of one or more mineral oil, Hydrogenated Polydecene, and/or Triglyceride, and 0% to 15 of a biologically active agent, and 0% to 30% of a tackifying agent, e.g., a hydrogenated ester of wood rosin.

In other embodiments, gelatinous elastomer compositions comprise 1-50% block copolymer, 10-70% triglyceride oil, 30-70% mid-block solubilizing oil, e.g., mineral or synthetic oil, 0-20% biologically active agent and 0-15% free fatty acids. In other embodiments, gelatinous elastomer compositions comprise 4-25% block copolymer, 10-70% triglyceride oil, 30-70% mid-block solubilizing oil, e.g., mineral or synthetic oil, 0-20% biologically active agent and 0-15% free fatty acids. In other embodiments, gelatinous elastomer compositions comprise 10-25% block copolymer, 10-70% triglyceride oil, 30-70% mid-block solubilizing oil, e.g., mineral or synthetic oil, 0-20% biologically active agent and 0-15% free fatty acids.

In other embodiments, gelatinous elastomer compositions comprise 1-50% block copolymer, 10-70% triglyceride oil, 40-60% mid-block solubilizing oil, e.g., mineral or synthetic oil, 0-20% biologically active agent and 0-15% free fatty acids. In other embodiments, gelatinous elastomer compositions comprise 4-25% block copolymer, 10-70% triglyceride oil, 40-60% mid-block solubilizing oil, e.g., mineral or synthetic oil, 0-20% biologically active agent and 0-15% free fatty acids. In other embodiments, gelatinous elastomer compositions comprise 10-25% block copolymer, 10-70% triglyceride oil, 40-60% mid-block solubilizing oil, e.g., mineral or synthetic oil, 0-20% biologically active agent and 0-15% free fatty acids.

In other embodiments, gelatinous elastomer compositions comprise 1-50% block copolymer, 20-60% triglyceride oil, 30-70% mid-block solubilizing oil, e.g., mineral or synthetic oil, 0-20% biologically active agent and 0-15% free fatty acids. In other embodiments, gelatinous elastomer compositions comprise 4-25% block copolymer, 20-60% triglyceride oil, 30-70% mid-block solubilizing oil, e.g., mineral or synthetic oil, 0-20% biologically active agent and 0-15% free fatty acids. In other embodiments, gelatinous elastomer compositions comprise 10-25% block copolymer, 20-60% triglyceride oil, 30-70% mid-block solubilizing oil, e.g., mineral or synthetic oil, 0-20% biologically active agent and 0-15% free fatty acids.

In other embodiments, gelatinous elastomer compositions comprise 1-50% block copolymer, 25-50% triglyceride oil, 40-60% mid-block solubilizing oil, e.g., mineral or synthetic oil, 0-20% biologically active agent and 0-15% free fatty acids. In other embodiments, gelatinous elastomer compositions comprise 4-25% block copolymer, 25-50% triglyceride oil, 40-60% mid-block solubilizing oil, e.g., mineral or synthetic oil, 0-20% biologically active agent and 0-15% free fatty acids. In other embodiments, gelatinous elastomer compositions comprise 10-25% block copolymer, 25-50% triglyceride oil, 40-60% mid-block solubilizing oil, e.g., mineral or synthetic oil, 0-20% biologically active agent and 0-15% free fatty acids.

In a preferred embodiment, the inventive gelatinous elastomer compositions generally comprise 5% to 30% of styrenic block copolymer TPE, and from 30% to 95% of an oil mixture having a triglyceride to total oil percentage from 3% to 60%. Further, these compositions may comprise from 0.0% to 20.0% of biologically active agent and 0% to 15.0% Free Fatty Acids. Gelatinous elastomer compositions may further comprise phytosterols, ceramides and/or bisabolol. Compositions may further comprise 0% to 30% of one or more tack modification agent. As discussed below, novel methods may optionally be employed to adjust and optimize inventive compositions within desired, and highly preferred, effective ranges. Particularly, optimization may be accomplished to assure desired rates of biologically active substance delivery to the body, as well as to adjust gel melt viscosity within ranges suitable to enable processing into articles of the invention.

The gels of the present invention may, for example, be made in mixers of various sizes depending on the amount of gel to be produced. In general, mixers similar to those employed for large scale batching of food stuffs (for example ribbon blending mixers made by Marion Mixers Inc.) which have been further outfitted with a thermostatically controlled heating jacket or heating elements to impart sufficient thermal energy to melt the polymer and aid in the blending of the materials as well outfitted with the ability to mix under a constant vacuum while at operating temperatures and mix speeds. Other suitable mixers are other heated mixing vessel such as heated vacuum polymer pots such as, for example, those available from ITWC Inc. outfitted with sufficient horsepower stirring or mixing blades to mix materials of high viscosity during heating. Typically, the liquid oil fraction or components are weighed out according to the desired formulation and placed into the heated mixing vessel and then brought to the specified temperature prior to weighing and adding the dry components, however, this order of addition is not specifically required for most formulations. In some embodiments, all components can be weighed out and added in any sequence, then heated and mixing accordingly. To aid mixing however, it is sometimes necessary to heat some amount of the oil component first, then add the polymers and other dry or solid components (such as tack resins, preservatives, pigments, fillers or other ingredients) slowly while mixing, then adding the remainder of the oil or liquid components (such as Mineral or Synthetic or Triglyceride oils, Vitamins or other additives, or fatty acids or esters or mono or di-glycerides as may be present in the formulation). In some preferred embodiments, mixing and blending temperatures range from about 100° C. to about 200° C., and more preferably are between about 130° C. and 180° C. depending on the specific chemistry and flash points of the oils employed or degradation temperatures of the other oils or additive components. Once all components to a mix formulation are added, vacuum is applied down to about 15 or more inches of Hg or sufficient level to prevent excess bubbles of air entrained in the gel, and blending then occurs at the preferred temperature and vacuum level for periods of, for example, between about 30 minutes and 10 hours, but preferably between about 1 hour and 5 hours, or until all components have been homogeneously blended and the gel is clear and free from lumps or agglomerations. From there, the gel can be dispensed into appropriate smaller containers for handling such as pails or steel drums whereby other melting equipment can be used to then re-melt the gel and transfer to the appropriate converting equipment.

Biologically Active Agents

In a preferred embodiment, the substance to be delivered by the gel is a biologically active agent with at least some solubility within the oil blend within the composition. For example, biologically active agents having cosmetic and or medicinal properties may be incorporated within these gel compositions and delivered to the body via oil exudation. Such agents are incorporated within the gel composition without limitation, preferably in an effective amount, so as to provide the desired level of therapeutic benefit effect.

Additionally, and preferably, various additives which are suitably soluble within the base oil composition may be incorporated within the inventive gel compositions for the purpose of inducing tack, enhancing the hand or feel of the material, and or to render the gel microbiologically static during storage and or use. Such agents are incorporated within the gel composition, for example and without limitation, in effective amounts, so arranged as to yield the desired level of associated effect.

Accordingly, gelatinous elastomer compositions can be used to deliver one or more biologically active agents. Biologically active agents include, for example and without limitation, pharmaceutical agents, pharmacological agents, biological agents, organic agents, natural agents, botanical agents, and cosmetic agents, e.g., agents for changing or improving skin, tissue or hair appearance, health or function.

Examples of biologically active agents include, for example and without limitation, Allantoin, Aloe Vera Oil, Alpha-Hydroxy Acid, Aluminum Hydroxide, Aspirin, Bacitracin, Benzoic Acid, Benzalkonium Chloride, Benzocaine, Beta-Hydroxy Acid, BHA, BHT, Bio Oil, Bisabolol, Bleomycim, Benzoic Acid, Boric Acid, Calcium Undecylenate, Calamine, Collagen, Camphor, Capric Acid, Caprylic Acid, Centella Asiatica, Ceramide 2, Ceramide 3, Ceramide 6, Chloral Hydrate, Clioquinol, Colloidal Oatmeal, Corticosteroids, Cyclomethicane Sulfate, Elderflower Extract, Emu Oil, Eugenol, Fouorouracil, Free Fatty Acids, Ferric Chloride, Ginkgo Biloba, Glycerin, Glycol Salicylate, Glycolic Acid, Glycosaminoglycans, Gotu kola, Grape Seed Extract, Helix Aspersa Muller Glycoprotein, Hexyresorcinol, Histamine dihydrochloride, Hyaluronic Acid, Hydrogen Peroxide, Imiquimod, Interferons, Linoleic Acid, Menthol, Menthoxypropanediol, Methyl Salicylate, Methylparaben, Miconasole Nitrate, Neomycin Sulfate, Oleic Acid, Oxyquinoline Sulfate, Panthenol, Penacycline triterpene resin, Phenol, Phenyl Salicylate, Povidone-vinylacetate copolymers, Propionic Acid, Propylparaben, Protein Hydrolysate, Purcellin Oil, Pyridoxine Hydrochloride, Quercetin, Resorcinol, Retinoic Acid, Retinol, Safflower oil, Salicylamide, Salicylic Acid, Silver Nitrate, Silver Ion, Simethicone, Sodium, Propionate, Sodium Salicylate, Sulfur, Tamanu Oil, Tamoxifin, Tannic Acid, Tea tree oil, Tetracycline Hydrochloride, Thymol, Tolindate, Tolnaftate, Topical Starch, Transforming Growth Factors, Trolamine, Trolamine Salicylate, Undecylenic Acid, Vitamin A Palmitate, Vitamin C, Vitamin D, Vitamin E Acetate, Zinc Acetate, Zinc Carbonate, Zinc Chloride, Zinc Oxide, Zinc Propionate, Zinc Sulfate, p-Menthane 3,8 diol Menthanediol, Octadecenadioic Acid, Glyceryl Hydrogenated Rosinate, Hydrogenated Gum Rosin, Pentaaerythrityl Hydrogenated Rosinate, Padinami Extract, Natural or Synthetic Ceramides (e.g., Ceramide BIO391, Synthetic Ceramides), Stearic Acid, Phytosterol, Lidocaine Hydrochloride.

Many other therapeutic agents can be incorporated into the gelatinous elastomeric compositions of the present invention. For example, antifungal agents (fungal agents) such as ciclopirox, chloroxylenol, undecylenic acid, tolnaftate, miconizole, clmibazole, clotrizole, griseofulvin, and ketoconozole may be incorporated therein. Antibiotic agents such as mupirocin, erythromycin, gentimycin, neomycin, polymyxin, bacitracin, tetracyclines, and the like may also be incorporated into the gelatinous composition. Antiseptic agents such as iodine, povidone-iodine, benzalkonium chloride, benzoic acid, chlorhexidine, nitrofurazone, benzoyl peroxide, hexachlorophene, phenol, resorcinol, and cetylpyridinium chloride likewise could be incorporated into the present invention. Furthermore, anti-inflammatories such as hydrocortisone, prednisone, triamcilolone, betamethasone and the like may be incorporated into the gelatinous composition. Still further, local anesthetics such as benzocaine, lidocaine, procaine, bupivicaine, a eutectic mixture of prilocaine and lignocaine, phenol, diphenhydramine, or the like may also be incorporated into the gelatinous composition. Additional agents that could be incorporated include penetration enhancers such as dimethyl sulfoxide or octolyphenylpolyethelene glycol, keratolytic agents such as salicylic acid, enzymes such as proteases and nucleases, hormones such as insulin, vesicants such as cantharadin, caustics such as podophyllin, and a many other additional pharmacologically active substances.

In some embodiments, examples of biologically active agents include, for example and without limitation, Allantoin, Aloe Vera Oil, Alpha-Hydroxy Acid, Aluminum Hydroxide, Aspirin, Bacitracin, Benzoic Acid, Benzalkonium Chloride, Benzocaine, Beta-Hydroxy Acid, BHA, BHT, Bio Oil, Bisabolol, Bleomycim, Benzoic Acid, Boric Acid, Calcium Undecylenate, Calamine, Collagen, Camphor, Capric Acid, Caprylic Acid, Centella Asiatica, Ceramide 2, Ceramide 3, Ceramide 6, Chloral Hydrate, Clioquinol, Colloidal Oatmeal, Corticosteroids, Cyclomethicane Sulfate, Elderflower Extract, Emu Oil, Eugenol, Fouorouracil, Free Fatty Acids, Ferric Chloride, Ginkgo Biloba, Glycerin, Glycol Salicylate, Glycolic Acid, Glycosaminoglycans, Gotu kola, Grape Seed Extract, Helix Aspersa Muller Glycoprotein, Hexyresorcinol, Histamine dihydrochloride, Hyaluronic Acid, Hydrogen Peroxide, Imiquimod, Interferons, Linoleic Acid, Menthol, Menthoxypropanediol, Methyl Salicylate, Methylparaben, Climbazole and all Conazole series fungicides such as Miconasole Nitrate, Neomycin Sulfate, Oleic Acid, Oxyquinoline Sulfate, Panthenol, Penacycline triterpene resin, Phenol, Phenyl Salicylate, Povidone-vinylacetate copolymers, Propionic Acid, Propylparaben, Protein Hydrolysate, Purcellin Oil, Pyridoxine Hydrochloride, Quercetin, Resorcinol, Retinoic Acid, Retinol, Safflower oil, Salicylamide, Salicylic Acid, Silver Nitrate, Silver Ion, Simethicone, Sodium, Propionate, Sodium Salicylate, Sulfur, Tamanu Oil, Tamoxifin, Tannic Acid, Tea tree oil, Tetracycline Hydrochloride, Thymol, Tolindate, Tolnaftate, Topical Starch, Transforming Growth Factors, Trolamine, Trolamine Salicylate, Undecylenic Acid, Vitamin A Palmitate, Vitamin C, Vitamin D, Vitamin E Acetate, Zinc Acetate, Zinc Carbonate, Zinc Chloride, Zinc Oxide, Zinc Propionate, Zinc Sulfate, p-Menthane 3,8 diol Menthanediol, Octadecenadioic Acid, Glyceryl Hydrogenated Rosinate, Hydrogenated Gum Rosin, Pentaaerythrityl Hydrogenated Rosinate, Padinami Extract, Natural or Synthetic Ceramides (e.g., Ceramide BIO391, Synthetic Ceramides), Stearic Acid, Phytosterol, Lidocaine Hydrochloride, Hydrolyzed Milk Protein, Urea, Octadecanadioic Acid (such as ODA-White® by Sederma) and other commercially available blends of the above including but not limited to for instance SymRepair™ by SymRise Corporation.

In an alternative embodiment, the biologically active agent is biologically active agent is a fungicide agent such as, for example, froaliphatic nitrogen fungicides: butylamine, cymoxanil, dodicin, dodine, guazatine, iminoctadine amide fungicides: carpropamid, chloraniformethan, cyflufenamid, diclocymet, ethaboxam, fenoxanil, flumetover, furametpyr, isopyrazam, mandipropamid, penthiopyrad. Prochloraz, quinazamid, silthiofam, triforine acylamino acid fungicides; benalaxyl, benalaxyl-M, furalaxyl, metalaxyl, metalaxyl-M, pefurazoate, valifenalate, anilide fungicides: benalaxyl, benalaxyl-M, bixafen, boscalid, carboxin, fenhexamid, isotianil, metalaxyl, metalaxyl-M, metsulfovax, ofurace, oxadixyl, oxycarboxin, penflufen, pyracarbolid, sedaxane, thifluzamide, tiadinil, benzanilide fungicides: benodanil, flutolanil, mebenil, mepronil, salicylanilide, tecloftalam, furanilide fungicides, fenfuram, furalaxyl, furcarbanil, methfuroxam, sulfonanilide fungicides, flusulfamide, benzamide fungicides: benzohydroxamic acid, fluopicolide, fluopyram, tioxymid, trichlamide, zarilamid, zoxamide, furamide fungicides, cyclafuramid, furmecyclox, phenylsulfamide fungicides: dichlofluanid, tolylfluanid, sulfonamide fungicides amisulbrom, cyazofamid, valinamide fungicides: benthiavalicarb, iprovalicarb, antibiotic fungicides, aureofungin, blasticidin-S, cycloheximide, griseofulvin, kasugamycin, natamycin, polyoxins, polyoxorim, streptomycin, validamycin, strobilurin fungicides, azoxystrobin, dimoxystrobin, fluoxastrobin, kresoxim-methyl, metominostrobin, orysastrobin picoxystrobin, pyraclostrobin, pyrametostrobin, pyraoxystrobin, trifloxystrobin, aromatic fungicides: biphenyl, chlorodinitronaphthalene, chloroneb, chlorothalonil, cresol, dicloran, hexachlorobenzene, pentachlorophenol, quintozene, sodium pentachlorophenoxide, tecnazene, benzimidazole fungicides: benomyl, carbendazim, chlorfenazole, cypendazole, debacarb, fuberidazole, mecarbinzid, rabenzazole, thiabendazole, benzimidazole precursor fungicides: furophanate, thiophanate, thiophanate-methyl, benzothiazole fungicides: bentaluron, benthiavalicarb, chlobenthiazone, probenazole, TCMTB, bridged diphenyl fungicides: bithionol, dichlorophen, diphenylamine, carbamate fungicides: benthiavalicarb, furophanate, iprovalicarb, propamocarb, pyribencarb, thiophanate, thiophanate-methyl, benzimidazolylcarbamate fungicides: benomyl, carbendazim, cypendazole, debacarb, mecarbinzid, carbanilate fungicides: diethofencarb, pyraclostrobin, pyrametostrobin, conazole fungicides: climbazole, clotrimazole, imazalil, ketoconazole, oxpoconazole, prochloraz, triflumizole, azaconazole, bromuconazole, cyproconazole, diclobutrazol, difenoconazole, diniconazole, diniconazole-M, epoxiconazole, etaconazole fenbuconazole, fluquinconazole, flusilazole, flutriafol, furconazole, furconazole-cis, hexaconazole, imibenconazole, ipconazole, miconazole nitrate, metconazole myclobutanil, penconazole, propiconazole, prothioconazole, quinconazole, simeconazole, tebuconazole, tetraconazole, triadimefon, triadimenol, triticonazole, uniconazole uniconazole-P, copper fungicides: Bordeaux mixture, Burgundy mixture, Cheshunt mixture, copper acetate, copper carbonate, basic copper hydroxide, copper naphthenate, copper oleate, copper oxychloride, copper silicate, copper sulfate, copper sulfate, basic copper zinc chromate, cufranebm, cuprobam, cuprous oxide, mancopper, oxine-copper, dicarboximide fungicides: famoxadone, fluoroimide, dichlorophenyl dicarboximide, fungicides: chlozolinate, dichlozoline, iprodione isovaledione, myclozolin, procymidone, vinclozolin, phthalimide fungicides: captafol, captan, ditalimfos, folpet, thiochlorfenphim, dinitrophenol fungicides: binapacryl, dinobuton, dinocap, dinocap-4, dinocap-6, meptyldinocap, dinocton, dinopenton, dinosulfon, dinoterbon, DNOC, dithiocarbamate fungicides: azithiram, carbamorph, cufraneb, cuprobam, disulfiram, ferbam, metam, nabam, tecoram, thiram, ziram, cyclic dithiocarbamate fungicides: dazomet, etem, milneb, polymeric dithiocarbamate fungicides: mancopper, mancozeb, maneb, metiram, polycarbamate, propineb, zineb, imidazole fungicides: cyazofamid, fenamidone, fenapanil, glyodin, iprodione, isovaledione, pefurazoate, triazoxide, inorganic fungicides: potassium azide, potassium thiocyanate, sodium azide, sulfur, inorganic mercury fungicides: mercuric chloride, mercuric oxide, mercurous chloride, organomercury fungicides: (3-ethoxypropyl)mercury bromide, ethylmercury acetate, ethylmercury bromide, ethylmercury chloride, ethylmercury 2,3-dihydroxypropyl, mercaptide, ethylmercury phosphate, N-(ethylmercury)-p-toluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriurea, phenylmercury acetate, phenylmercury chloride, phenylmercury derivative of pyrocatechol, phenylmercury nitrate phenylmercury salicylate, thiomersal, tolylmercury acetate, morpholine fungicides: aldimorph, benzamorf, carbamorph, dimethomorph, dodemorph, fenpropimorph, flumorph, tridemorph, organophosphorus fungicides: ampropylfos, ditalimfos, edifenphos, fosetyl, hexylthiofos, iprobenfos, phosdiphen, pyrazophos, tolclofos-methyl, triamiphos, organotin fungicides: decafentin, fentin, tributyltin oxide, oxathiin fungicides: carboxin, oxycarboxin, oxazole fungicides: chlozolinate, dichlozoline, drazoxolon, famoxadone, hymexazol, metazoxolon, myclozolin, oxadixyl, vinclozolin, polysulfide fungicides: barium polysulfide, calcium polysulfide, potassium polysulfide, sodium polysulfide, pyrazole fungicides: bixafen, furametpyr, isopyrazam, penflufen, penthiopyrad, pyraclostrobin, pyrametostrobin, pyraoxystrobin, rabenzazole, sedaxane, pyridine fungicides: boscalid, buthiobate, dipyrithione, fluazinam, fluopicolide, fluopyram, pyribencarb, pyridinitril, pyrifenox, pyroxychlor, pyroxyfur, pyrimidine fungicides: bupirimate, diflumetorim, dimethirimol, ethirimol, fenarimol, ferimzone, nuarimol, triarimol, anilinopyrimidine fungicides: cyprodinil, mepanipyrim, pyrimethanil, pyrrole fungicides: fenpiclonil, fludioxonil, fluoroimide, quinoline fungicides: ethoxyquin, halacrinate, 8-hydroxyquinoline sulfate, quinacetol, quinoxyfen, tebufloquin, quinone fungicides: benquinox, chloranil, dichlone, dithianon, quinoxaline fungicides: chinomethionat, chlorquinox, thioquinox, thiazole fungicides: ethaboxam, etridiazole, isotianil, metsulfovax, octhilinone thiabendazole, thifluzamide, thiazolidine fungicides: flutianil, thiadifluor, thiocarbamate fungicides: methasulfocarb, prothiocarb, thiophene fungicides: ethaboxam, silthiofam, triazine fungicides: anilazine, triazole fungicides: amisulbrom, bitertanol, fluotrimazole, triazbutil triazolopyrimidine fungicides: ametoctradin, urea fungicides: bentaluron, pencycuron, quinazamid, urea, unclassified fungicides: acibenzolar, acypetacs, allyl alcohol, benzalkonium chloride, benzamacril, bethoxazin, carvone, chloropicrin, DBCP, dehydroacetic acid, diclomezine, diethyl pyrocarbonate, fenaminosulf, fenitropan, fenpropidin, formaldehyde, furfural, hexachlorobutadiene, iodomethane, isoprothiolane, methyl bromide, methyl isothiocyanate, metrafenone, nitrostyrene, nitrothal-isopropyl, OCH, 2-phenylphenol, phthalide, piperalin, proquinazid, pyroquilon, sodium orthophenylphenoxide, spiroxamine, sultropen, thicyofen, tricyclazole, zinc naphthenate.

In some embodiments, the compositions of the present invention comprise up to 20% or even up to 40% of any biologically active agent which may be dissolved, at some level, within the gel composition and which may, for whatever purpose, be useful for delivery to the body. Such biologically active agents include, for example and without limitation, pharmaceutical agents, pharmacological agents, biological agents, organic agents, natural agents, botanical agents, and cosmetic agents, e.g., agents for changing or improving skin, tissue or hair appearance, health or function. With the scope of the present inventive concept, such agents may serve any therapeutic purpose upon application in contact with any part of the body including, for example and without limitation, skin, hair, teeth, body orifices such as the mouth, rectum and vagina, and even internally such as on the surface of catheters, stents, and the like.

In certain preferred embodiments, an effective amount of a therapeutically active formulation comprising a vitamin additive is incorporated into the gelatinous/plasticizing oil mixture. The vitamin additive is, for example, selected from Vitamin A, B₁₂, C, D, E, and mixtures thereof. Preferably, the vitamin additive is present in the therapeutically active formulation at a concentration of, by weight percent, about 1% to about 10%.

In certain embodiments, gelatinous elastomer compositions comprise salicylic acid. Salicylic acid may be added to formulations by combining it in equal parts with ceramide-3 at a temperature above the melting point of salicylic acid, but below its degradation temperature, to form a homogeneous liquid, cooling the liquid to a waxy solid, and then combining the solid with the oil portion of the gel.

In other embodiments, gelatinous elastomer compositions comprise quercetin. Quercertin may be added to formulations by first blending with one or more of ceramide-3, DP-70 mineral oil, hydrogenated polydecene, or coconut oil.

In some embodiments, biologically active agents are generally comprise a total 0-20% of a compositions (wt/wt).

Compositions may further comprise 0% to 5.0% Free Fatty Acids, phytosterols and ceramides, and/or bisabolol.

In a preferred embodiment, the gelatinous elastomer compositions of the present invention comprise from about 50% to about 80% by weight of a hydrogenated polydecene, from about 20% to about 50% by weight of a Cocos nucifera (Coconut) Oil, from about 5% to about 19% by weight of a hydrogenated styrene-ethylene/butylene-styrene copolymer, from about 1% to about 10% by weight of a hydrogenated styrene isoprene/butadiene copolymer; from about 2% to about 20% by weight of a hydrogenated styrene isoprene/butadiene copolymer; and, optionally, from about 1% to about 10% by weight of a vitamin E source, preferably tocopheryl acetate.

In another preferred embodiment, the gelatinous elastomer compositions of the present invention comprise from about 50% to about 80% by weight of a hydrogenated polydecene, from about 7% to about 25% by weight of a Cocos nucifera (Coconut) Oil, from about 5% to about 19% by weight of a hydrogenated styrene-ethylene/butylene-styrene copolymer, from about 1% to about 10% by weight of a hydrogenated styrene isoprene/butadiene copolymer; from about 2% to about 20% by weight of a hydrogenated styrene isoprene/butadiene copolymer; and, optionally, from about 1% to about 10% by weight of a vitamin E source, preferably tocopheryl acetate, from about 1% to about 10% by weight of a Prunus Amygdalus Duclis (Non-GMO Sweet Almond) Oil, and from about 1% to about 10% by weight of a Bertholletia excelsa (Community Trade Brazil) Nut Oil.

In another preferred embodiment, the gelatinous elastomer compositions of the present invention comprise from about 50% to about 80% by weight of a mineral oil (Paraffinum Liquidum), from about 20% to about 50% by weight of a Hydrogenated Styrene Isoprene Copolymer, from about 2% to about 20% by weight of a hydrogenated styrene isoprene/butadiene copolymer, from about 1% to about 10% by weight of a camphor resin; from about 1% to about 10% by weight of a hydrocarbon resin; from about 1% to about 10% by weight of a Hydrogenated Ester of Wood Rosin and, optionally, from about 1% to about 10% by weight of menthol.

In another preferred embodiment, the gelatinous elastomer compositions of the present invention comprise from about 50% to about 80% by weight of a hydrogenated polydecene, from about 7% to about 25% by weight of a Cocos nucifera (Coconut) Oil, from about 5% to about 19% by weight of octyl palmitate, from about 5% to about 19% by weight of safflower oil, from about 6% to about 29% by weight of a Hydrogenated Styrene-Ethylene/Butylene-Styrene Copolymer, from about 1% to about 10% by weight of a fractionated coconut oil; from about 2% to about 20% by weight of a Hydrogenated Styrene Isoprene/Butadiene Copolymer; and, optionally, from about 1% to about 10% of a vitamin A source, preferably Menthanediol (p-menthane 3,8 diol).

In another preferred embodiment, the gelatinous elastomer compositions of the present invention comprise from about 20% to about 50% by weight of a hydrogenated polydecene, from about 7% to about 25% by weight of a Hydrogenated Styrene-Isoprene-Styrene Copolymer, from about 3% to about 30% by weight of a Hydrogenated Styrene-Ethylene/Butylene-Styrene Copolymer, from about 2% to about 20% by weight of a Hydrogenated Styrene Isoprene/Butadiene Copolymer, from about 1% to about 10% by weight of a fractionated coconut oil; from about 1% to about 10% by weight of safflower oil, and, optionally, from about 1% to about 10% of a vitamin A source, preferably Vitamin A Palmitate (Retinyl Palmitate).

In another preferred embodiment, the gelatinous elastomer compositions of the present invention comprise from about 27% to about 75% by weight of a mineral oil (Paraffinum Liquidum), from about 15% to about 35% by weight of a Hydrogenated Styrene Isoprene Copolymer, from about 2% to about 20% by weight of a hydrogenated styrene isoprene/butadiene copolymer, from about 1% to about 10% by weight of a hydrocarbon resin; from about 1% to about 10% by weight of a Glyceryl Hydrogenated Rosinate and, optionally, from about 1% to about 10% of a vitamin A source, preferably Vitamin A Palmitate (Retinyl Palmitate).

In another preferred embodiment, the gelatinous elastomer compositions of the present invention comprise from about 20% to about 50% by weight of a hydrogenated polydecene, from about 20% to about 50% by weight of a Cocos nucifera (Coconut) Oil, from about 6% to about 29% by weight of a Hydrogenated Styrene-Ethylene/Butylene-Styrene Copolymer, from about 1% to about 10% by weight a wheat germ oil, from about 2% to about 20% by weight of a Hydrogenated Styrene-Ethylene/Butylene-Styrene Copolymer, from about 1% to about 10% by weight of a fractionated coconut oil; from about 2% to about 20% by weight of a Hydrogenated Styrene Isoprene/Butadiene Copolymer; and, optionally, from about 1% to about 10% of a of a vitamin E source, preferably tocopheryl acetate.

In another preferred embodiment, the gelatinous elastomer compositions of the present invention comprise from about 50% to about 80% by weight of a hydrogenated polydecene, from about 7% to about 25% by weight of a Cocos nucifera (Coconut) Oil, from about 5% to about 19% by weight of a Hydrogenated Styrene-Ethylene/Butylene-Styrene Copolymer, from about 1% to about 10% by weight a wheat germ oil, from about 7% to about 39% by weight of a Hydrogenated Styrene Isoprene/Butadiene Copolymer; from about 1% to about 10% by weight of a Persea gratissima (Avocado) Oil; from about 1% to about 10% by weight of a hydrocarbon resin; from about 1% to about 10% by weight of a Hydrogenated Ester of Wood Rosin, and, optionally, from about 1% to about 10% of a of a vitamin E source, preferably tocopheryl acetate.

In another preferred embodiment, the gelatinous elastomer compositions of the present invention comprise from about 50% to about 80% by weight of a hydrogenated polydecene, from about 7% to about 25% by weight of a Cocos nucifera (Coconut) Oil, from about 6% to about 29% by weight of a Hydrogenated Styrene-Ethylene/Butylene-Styrene Copolymer, from about 5% to about 19% by weight a safflower oil, from about 2% to about 10% by weight of a Hydrogenated Styrene Isoprene/Butadiene Copolymer; and from about 1% to about 10% by weight of a Fractionated Coconut Oil.

In another preferred embodiment, the gelatinous elastomer compositions of the present invention comprise from about 20% to about 50% by weight of a mineral oil (Paraffinum Liquidum), from about 20% to about 50% by weight of a Hydrogenated Styrene Isoprene Copolymer, from about 1% to about 10% by weight of a Melaleuca alterniflora (Tea Tree) Oil, and from about 20% to about 50% by weight of a hydrocarbon resin.

Articles of the Present Invention

In a another aspect of the invention, articles are provided which enable a user to suitably apply the inventive gel compositions to human or animal bodies for the purpose of delivering active biological agents. Such articles take the form of molded inventive gel pads, patches, cylinders, tubes, orifice/body contour shaped patches/plugs, and wearable fabric articles coated with inventive gel compositions.

The compositions described herein may be molded as independent stand-alone articles to be worn in contact with body tissue or skin or hair, or molded as composite articles with, for example and without limitation, pre-formed gloves, socks, booties, cuffs, sleeves, bands, belts, pads, cylinders, patches, socks, leggings, pants, undergarments, or internal body cavity devices specifically designed to deliver portions of the composition to the skin, body tissue or hair. The compositions may also be molded as composite articles with polymeric and/or organic substrate films, non-woven webs, or woven fabrics that can be cut to specific sizes, shaped or shaped into articles or patches. Such articles may be constructed to form a direct delivery system for a biologically active agent, such that when they are applied the gelatinous composition is in direct contact with body tissue, skin or hair, thus providing for direct topical delivery of biologically active agents included in the composition. Alternatively articles may be constructed to form an indirect delivery system wherein a permeable membrane is interspersed between the gelatinous composition and a body tissue, skin or hair.

In an additional embodiment of the present invention, any of the gel compositions outlined above, may be utilized in a method for delivery of biologically active substances to the body comprising the steps of preparing inventive gels in the form of an article suitable for wearing or applying on or within the body, and applying this form/article for a time sufficient to effect the desired dose delivery. Preferred articles of this type include molded inventive gel pads, patches, cylinders, tubes, orifice/body contour shaped plugs/patches, and wearable fabric articles coated comprising the inventive gel compositions.

The specific active ingredients employed utilizing the gelatinous elastomer as both a reservoir and carrier to control the rate of release represents a novel method of treatment when employed as molded articles, gloves, socks, booties, cuffs, sleeves, bands, belts, pads, cylinders, patches, socks, leggings, pants, undergarments, or internal body cavity devices specifically designed to deliver portions of the composition to the skin, body tissue or hair. Among the specific indications of use for these formulations, depending on the biologically active agent included therein, are skin softening, cosmetic enhancement, lipid barrier improvement, wrinkle reduction, skin smoothing, scar reduction, scar management, stretch mark reduction, stretch mark management, anti-histamine, anti-inflammatory, anti-oxidant, anti-microbial, anti-arthritic, acne treatment, muscle relaxation, aromatherapy, soft tissue conditioning, skin elastase improvement, cell repair, skin cooling, skin warming, hair conditioning, hair strength improvement, hair cosmetic enhancement, lip plumping, counter-irritation, burn treatment and pain relief.

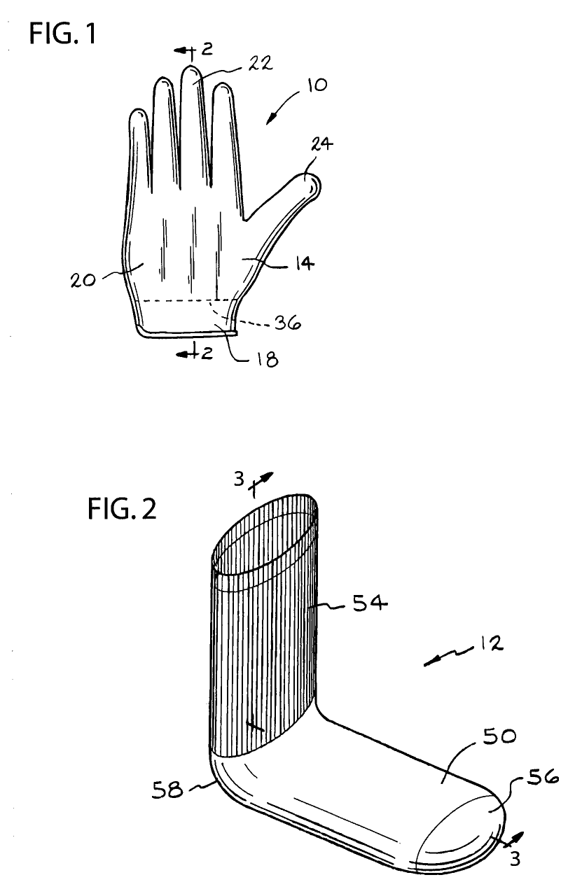

Turning now to the drawings, FIGS. 1 AND 2 show various embodiments of body protection articles constructed according to the principles of the present invention. FIG. 1 shows a glove 10 and FIG. 2 illustrates a sock 12. However, those skilled in the art will readily appreciate that the glove 10 and sock 12 shown are only exemplary, and many different articles worn on the body are useful for imparting a therapeutically active formulation to the covered skin delivered from a gelatinous elastomeric composition according to the present invention. In a broader sense, however, a body protective article is provided in any shape and size required to cover a particular body part including shaped pads for use by women, men and children of all ages and sizes.

As shown in FIG. 1, the glove 10 is comprised of a palm piece 14 and a backhand piece 16, each a mirror image of the other. The palm piece 14 includes a wrist portion 18 extending across a palm portion 20 to four finger extensions 22 and a thumb extension 24. The back piece 16 of the glove similarly has a wrist portion 26 extending across a backhand portion 28 to form finger extensions 30 and a thumb extension (not shown). The palm piece 14 and the backhand piece 16 are joined together at their peripheral edges, such as by sewing, except at the respective wrist portions 18 and 26 providing an opening for putting a hand in the glove. A wrist piece 32 is folded over onto both sides of the palm piece 14 and the backhand piece 16 and sewn thereto surrounding the glove opening to prevent fraying and to add integrity for pulling the glove 10 onto a hand and for removing it therefrom.

The palm piece 14 and the backhand piece 16 are made from a cloth material having a gelatinous elastomeric composition 34 intimately bonded thereto. The gelatinous composition 34 extends from a location spaced from the wrist piece 32, as shown by the dashed line 36, to the ends of the fingers 22, 30 and the thumb 24. Preferably the inner surface of the gelatinous composition closest to the human hand wearing the glove 10 directly contacts the skin.

The cloth material can be a textile fabric constructed of either or both of a synthetic or natural fiber. Suitable synthetic materials includes fibers such as polyester, polyamide such as nylon, polyolefin, acrylic and like fibers while suitable natural fibers include cotton, cambric, wool, cashmere, rayon, jute and others.

FIG. 2 shows a sock 12 according to another embodiment of the present invention. The sock 12 is comprised of foot portion 50 leading to an ankle portion 52 extending to a lower leg portion 54. The sock 12 can be made having a generally tubular construction closed at one end by a toe portion 56 and seamed to provide a heel recess 58. In a similar manner as the glove 10, the sock 12 is made from a knitted cloth having a gelatinous elastomeric composition 60 intimately bonded thereto. The cloth and gelatinous composition of the sock 14 are selected from materials similar to those used to construct the respective palm and backhand pieces 14, 16 and the gelatinous composition 34 of the glove 10.

The gelatinous material preferably directly contacts the skin in a similar manner as shown and described with respect to the glove 10 to medicate the protected skin by means of a therapeutically active formulation as an additive incorporated therein. The gelatinous composition extends from the toe portion to the heel and has a width sufficient to cover the bottom of the foot.

Thus, the present invention molded and/or flexible articles are formed from a molten blend of the gelatinous elastomeric composition of the present invention, optionally comprising an active agent, intimately bonded to a cloth, fabric, paper or a polymeric film substrate by blending, melting, dipping, casting, injection molding, extruding and other conventional methods. For example, a preselected rigidity of a molten gelatinous elastomer composition is cast directly onto a cloth material to form the molded or flexible article such as glove 10 and sock 12. The gelatinous elastomer composition can also be die cast, cut to size and heat bonded to the substrate. Likewise, a substrate such as of a cloth, paper, or a polymeric film material can be dipped into a preselected rigidity of a molten gelatinous elastomer composition and re-dipped into the same or different composition of a different rigidity. The shaped composite article of the invention can be conventionally covered with protective skins of elastomeric film, paper, cloth, fabric or combinations thereof, as needed.

Methods of Controlling Exudation Rate and Delivery Rate of Active Substances

Among the novel methods of the present invention is the ability to adjust the exudation rate of the oils from the gel to achieve a rate such that the gel may be used to deliver a beneficial amount of other ingredients that have been shown to be compatible with, miscible in and deliverable to the body. In ratios of mid-block solubilizing oil to triglyceride oil of about 99 to 1 and higher, the amount of triglyceride only has a minimal effect on the exudation rate of oil from the gel as compared to a gel which only contains a mid-block solubilizing oil. In ratios mid-block solubilizing oil to triglyceride oils of about 90, 91, 92, 93, 94, 95, 96 up to 97 or 98 to 10, 9, 8, 7, 6, 5, 4, 3, or 2, the exudation rate, and corresponding active agent delivery rate, increases. In some embodiments, when ratio of mid-block solubilizing oil to triglyceride oil is from about 60:40 to about 40:60 the gel composition can deliver from about 300% to 800% more active agent to a surface, e.g., the human or animal body, than a comparable gel composition containing no triglyceride oil or a very low level of triglyceride (i.e., that having a mid-block solubilizing oil to triglyceride oil ratio of 200:1 and less). As demonstrated above in Table I (below), the choice of the mid-block solubilizing oil to triglyceride oil ratio should be balanced against the physical properties of the gel for both end use and for ease of processing due to viscosity of the melt.

Increasing the total oil weight percent and decreasing the total polymer weight percent can achieve a higher exudation rate from a gel, however, the physical properties of the gel are such that it is often too soft and weak for the intended end use. This is especially true of tear strength. Also, some ingredients are not readily soluble in mid-block solubilizing oils alone, and are more readily soluble in oil mixtures containing mid-block solubilizing oils and oils containing, e.g., monoglycerides, diglyceride and/or triglycerides (e.g., the triglyceride oil of the present invention). Thus, the gel compositions of the present invention allow for increased delivery of an active agent without compromising the physical properties of the gel, and also allow for the solubilization of a larger class of active agents than gels containing only a mid-block solubilizing oil (e.g., active agents of high polarity).

Additionally, use of specific a monoglycerides, diglyceride or triglycerides in combination with specific MW and midblock construction copolymers can lead to the ability to balance additional ingredient solubility, with mix viscosity at processing temperatures (i.e., temperatures in the melting range of the polymers), exudation rate of the resultant gel, and also end physical properties of the resultant gel. In some embodiments, useful molten viscosities for processing (i.e. viscosity of molten gel at processing temperature) range from about 80 to about 4000 cPs at 375° F. (190° C.), preferably from about 150 to about 3000 cPs at 375° F. (190° C.) and more preferably from about 190 to about 2500 cPs at 375° F. (190° C.).

The aforementioned molten viscosities can be measured using a #27 spindle at 150 to 200 RPM with Brookfield Viscometer model DVII+ with Brookfield Thermocell. Other viscometers and viscosity standards which can be converted to centipoise scales can be substituted as suitable measurement criteria. Below a viscosity of about 190 cPs gel tends to bleed through most substrates, especially textile substrates. Above a viscosity of about 2500 cPs, gel tends to be too thick for a number of conversion processes such as dipping and laminating, but may still be useful for injection molding.

Most preferred, however, are levels of the triglyceride oil ratio which are optimized to yield a desired rate of biologically active substance delivery upon contact between the gel and the body. Methods for such optimization include any technique whereby the rate of exudation/diffusion of biologically active material produced by a given gel composition, in contact with the body or some other representative material, is measured, and alternative gel compositions having different levels of triglyceride oil ratio are iteratively tested, until a desired delivery rate is achieved. Associated methods may comprise exudation testing, as described in greater detail below, wherein gel samples are exposed to a non-living substance of some type, allowed to function for a period of time, removed from contact, exudate is extracted from the non-living substance, and exudate is analyzed to determine the level of actual bioactive substance delivery. Multivariate optimizations, wherein the level of bioactive substance within the gel, styrenic block-copolymer TPE composition, and triglyceride oil ratio, are all varied systematically, are also within the scope of related inventive methodology.

As mentioned above, it is most preferred to optimize inventive compositions using test methods aimed at quantification of exudation rates, in order to yield a desired rate of biologically active substance delivery. Although seemingly straightforward, the application of such technique is generally hindered by complexities associated with mimicking the body for purposes of measuring topical delivery. Since it is difficult, and sometimes virtually impossible, to measure the true quantity of a bioactive substance actually delivered by a gel to a living subject, it is often necessary to either measure and optimize indirect clinical effects on living subjects (requiring great care, expense, and replication), or to devise a measurement which, in some way, correlates with exudation in vivo. Such techniques are contemplated within the methods of the present invention.

Notwithstanding the difficulty of measuring the actual quantity of bioactive substance delivery to the body, the methods outlined herein are preferred for the purposes of gel optimization (whenever possible). Specifically, where analytical tests may be performed on blood, urine, bodily fluids, or bodily tissue as a means to gage level of delivery to the body over time, the functionality of a given gel composition may be quantified in actual use. Then, via iterative adjustment of gel composition, particularly the mid-block solubilizing oil to triglyceride oil ratio and percentage of bioactive substance within the gel, actual delivery rate may be optimized. Through this novel method, effective levels for the both the mid-block solubilizing oil to triglyceride oil ratio and bioactive percentage within the gel may be determined (effective levels being defined as levels so arranged as to produce a desired mean rate of active agent delivery to a population of live subjects under specified conditions). Accordingly, optimization of the mid-block solubilizing oil to triglyceride oil ratio, and active agent weight percentage, via such direct in vivo measurement methodology is contemplated by the methods disclosed herein.

In another embodiment of the present invention, any of the gel compositions outlined above may be optimized via a method wherein the composition containing the components within the general weight percent ranges provided above, is varied in order to achieve a desired rate of controlled oil delivery. One associated method comprises the steps of identifying a desired rate of topical delivery of an active substance from the gel into/onto a substance mimicking the body, pressing samples of gel into the substance, measuring the rate of oil/bioactive substance delivery to the substance, and varying the mid-block solubilizing oil to triglyceride oil ratio within subsequent formulations of the gel composition until a desired level of exudation is achieved. Another associated method comprises the steps of identifying a desired clinical effect on the human or animal body (or upon a condition thereof), performing clinical studies of actual effect of the gel on a population of subjects (in vivo), and varying the gel composition iteratively, e.g., by modifying the mid-block solubilizing oil to triglyceride oil ratio, to achieve an effective level of active biological substance delivery.

In another embodiment of the present invention, any of the gel compositions outlined above may be optimized via a method wherein the composition containing the components within the general weight percent ranges provided above, is varied in order to achieve a molten viscosity desired for the sake of melt processing at a desired temperature. This method comprises the steps of identifying a desired level for a viscosity dependent processing parameter (e.g. the level of wicking into or through fabric during dip coating) and varying, e,g, mid-block solubilizing oil to triglyceride oil ratio in subsequent samples of the gel (within the general ranges set forth above) until the desired processing parameter level is achieved.

In a preferred embodiment of the present invention, gel compositions are optimized through practice of two or more of the above variation methods for optimization of delivery rate, and melt viscosity, as set forth above.

A less direct, though preferred technique for the optimization of bioactive delivery rates associated with inventive gel compositions involves application of gel to a population of living subjects under controlled conditions, and measurement of gel impact on conditions of interest (or conditions targeted for therapeutic treatment). Through this novel method, gel composition, particularly the triglyceride oil level, and percentage of bioactive substance with the gel, may then be iteratively optimized to achieve effective levels (effective levels being levels so arranged as to produce a desired level of clinical outcome). Accordingly, optimization of gel triglyceride ratio, and bioactive percentage, via such clinical study of gel effects on conditions within live subjects, is considered a facet of the overall inventive concept disclosed herein.

An even less direct, but still preferred method for the optimization of bioactive delivery rates associated with inventive gel compositions involves application of gel to a non-living substance, followed by direct analytical measurement of actual delivery rates. While a wide range of substances may be used for such purposes, including other gels, and substances designed in various ways to mimic the chemical/physical behavior of living tissue, relatively simple substrates, including paper, may also be employed. Whatever the substrate, however, actual bioactive substance delivery therein, cannot be guaranteed to exactly mimic that of gel in contact with an actual living body (in fact, direct correspondence is not to be expected). Nonetheless, generally monotonic behavior may be expected, whatever the test substrate, and should correlate in some way with actual in vivo rates. Thus, the relative performance of gels may be evaluated and optimized. With study, and through examination of relative performance of gels both against a substrate, and in vivo, both the triglyceride oil ratio, and gel percentage of bioactive substances, may be adjusted to achieve a desired level (as determined via some correlation with in vivo observation).

While less direct, this type of method actually has the advantage of being capable of producing highly analytical results insofar as relative measurement of gel exudation is concerned. Since non living substrates may be chemically analyzed in any desirable manner (including via solvent extraction), it is possible to use this technique to carry out extremely precise measurement of bioactive exudation rate within a highly systematic framework. Using pure substrates such as filter paper, for example, exudates may be extracted into a known quantity of solvent, and subsequently analyzed via techniques including, for example and without limitation, chemical assay, titration, gas chromatography (GC), and high performance liquid chromatography (HPLC), in order to determine the level and rate of delivery (at least within the test system). Relative comparisons among gels having different bioactive substance percentage, and different triglyceride oil ratios, may then be carried out with analytical precision. In addition, manipulation of these compositional parameters so as to achieve a desired rate of delivery (especially where any in vivo cross comparison exists for any comparative benchmark gel composition), may be carried out.

While such a method of compositional optimization is less direct than clinical approaches, is has the advantage of being far more analytically precise, and more easily controlled. For example, gels may be applied to a substrate (including, without limitation, materials such as filter paper), for any desired period, at any desired/controlled temperature, and under extremely well controlled stress conditions (with, for example and without limitation, an external load applied via dead weight). Additionally, large numbers of replicate measurements are possible, with analytical precision, without the effort and expense associated with clinical studies on live subjects. In fact, this novel method was specifically developed for the purpose of, and used extensively in, overall development of inventive gel compositions. Accordingly, the associated method of optimizing gel composition is considered within the scope of the overall inventive concept disclosed herein.

Through the novel measurements and methods outlined above, it becomes possible to gage the effectiveness of a gel composition, and effect the desired level of bioactive delivery rate and or associated effect (via manipulation of the bioactive concentration within the gel as well as the triglyceride oil ratio). Thus, it becomes possible to actually employ the gel, in contact with human or animal bodies, for a such a time that the desired dose of bioactive substance is delivered, given a known area of gel contact.

### EXAMPLES

### Example 1

### Exemplary Gel Composition Base Formulations

The following are exemplary gel composition base formulations which can then be combined with, for example, from about 0% to about 5% by weight of the one or more pharmaceutical and cosmetic active ingredients described above, including salicylic acid, methyl salicylate, menthoxypropanediol, natural menthol-L, quercetin, and ceramide-3.

| | Formulae | | | | |
|---|---|---|---|---|---|
| | 06-087B | 06-087CS | D1028B | D1030B | S22B |
| Kraton 1654 SEEPS Polymer | 9.000% | 9.000% | | | 7.000% |
| Septon S4055 Polymer | 3.000% | 3.000% | | | 7.000% |
| Septon S4033 Polymer | 2.000% | 2.000% | | | |
| Kraton RP6935 Polymer | | | 16.750% | 16.750% | |
| Duoprime 70 Mineral Oil | | | | | |
| 76 deg. Coconut Oil | 33.800% | 33.500% | 32.000% | 32.000% | 29.800% |
| Jojoba Oil | | | | | 28.000% |
| Octyl Palmitate | | | | 16.657% | 27.000% |
| Exon Mobil Pure Syn 2 | 51.607% | 51.500% | 16.657% | | |
| Ultra Refined Sesame Oil | | | 16.750% | 16.750% | |
| 70/30 Capric/Caprilyc Triglycerides | | | 16.750% | 16.750% | |
| Fragrance | 0.075% | 0.000% | 0.075% | 0.075% | 0.100% |
| Vit E Act. | 0.500% | 1.000% | 1.000% | 1.000% | 1.000% |
| BHT | 0.018% | 0.000% | 0.018% | 0.018% | 0.100% |
| TOTAL | 100.000% | 100.000% | 100.000% | 100.000% | 100.000% |

Base Formulation

Similar formulations that exhibit soft elastic behavior and sufficient tack to be self-adhesive are obtaining by addition of 5% to 25% of various blends of Regelrez 1094, Foral AX, Foral 85-E or H100-W or similar natural or synthetic rosins as a tack inducing ingredient, e.g., 5% to 25% of a 50:50 blend of Regalrez 1094 and Foral AX.

Other exemplary gel composition base formulations include the following:

| Component | SCM001 | SCM002 | SCM003 | SMT1031 | SCM501 |
|---|---|---|---|---|---|
| Pure Syn 2 (Hydrogenated Polydecen) | 51.00% | 51.50% | 52.75% | 48.00% | 35.00% |
| Octyl Palmitate | 7.00% | 7.00% | 7.00% | | 8.00% |
| Coconut Oil | 14.76% | 13.26% | 13.26% | | |
| MCT Oil 70/30 (Capric/Caprylic | 4.50% | 4.00% | 4.00% | 16.00% | 9.00% |
| Triglycerides) | | | | | |
| High Linoleic Safflower Oil | 5.00% | 5.00% | 5.00% | | 2.00% |
| Vitamin A Palmitate | 1.00% | 1.00% | 1.00% | | 2.00% |
| SymRise SymRepair (TM) 153884 | 0.50% | 0.50% | 0.50% | | 1.00% |
| Quercetin Extract | 0.20% | 0.20% | 0.20% | | 1.50% |
| Ceramide 3 | 1.00% | 0.50% | 0.50% | | 0.40% |
| p-Menthane 3,8 diol (Lipo Coolact 38D) | 0.50% | 0.75% | 0.75% | | 0.50% |
| Vitamin E Acetate | 0.50% | 0.50% | 0.50% | | 0.50% |
| Propyl Paraben | 0.02% | 0.02% | 0.02% | | 0.05% |
| BHT | 0.02% | 0.02% | 0.02% | | 0.05% |
| Kraton D1117-BT | | | | 24.00% | 17.00% |
| Kraton G1654 | 4.50% | 6.50% | 6.00% | | |
| Kraton G1641H | | | | 8.00% | 5.00% |
| Kraton MD6945-10 | 5.00% | 4.00% | | | 5.00% |
| Kraton RP6935 | | | 3.50% | | |
| Septon 2063 | | | | | 4.00% |
| Septon 4055 alternate Kraton G1651H | 2.75% | 3.25% | 3.00% | | 2.00% |
| Septon 4033 alternate Kraton G1650 | 1.75% | 2.00% | 2.00% | | |
| Eastman Regalrez 1094 | | | | | 2.00% |
| Eastman Foral AX Rosin | | | | | 2.00% |
| Eastotac H100-W Rosin | | | | 4.00% | 3.00% |
| | 100.00% | 100.00% | 100.00% | 100.00% | 100.00% |

| Component | 06-087CSR2 | SCM501B | SCM502 | SCM503 | SCM504 |
|---|---|---|---|---|---|
| Pure Syn 2 (Hydrogenated Polydecen) | 51.485% | 43.24% | 43.75% | 43.50% | 43.85% |
| Octyl Palmitate | | 4.00% | 10.00% | 7.00% | 7.00% |
| Coconut Oil | 33.50% | 16.75% | 3.00% | 10.00% | 9.00% |
| MCT Oil 70/30 (Capric/Caprylic | | 4.50% | 4.00% | 4.50% | 4.50% |
| Triglycerides) | | | | | |
| High Linoleic Safflower Oil | | 1.00% | 1.00% | 1.00% | 1.00% |
| Vitamin A Palmitate | | 1.00% | 1.00% | 1.00% | 1.00% |
| SymRise SymRepair (TM) 153884 | | 0.50% | 0.50% | 0.50% | 0.50% |
| Quercetin Extract | | 0.75% | 0.50% | 0.30% | 0.20% |
| Ceramide 3 | | 0.20% | 0.21% | 0.21% | 0.16% |
| p-Menthane 3,8 diol (Lipo Coolact 38D) | | 0.25% | 0.50% | 0.50% | 0.50% |
| Vitamin E Acetate | 1.00% | 0.75% | 0.50% | 0.70% | 0.50% |
| Propyl Paraben | | 0.03% | 0.02% | 0.02% | 0.02% |
| BHT | 0.015% | 0.03% | 0.02% | 0.02% | 0.02% |
| Kraton D1117-BT | | 8.50% | 12.00% | 8.50% | 8.50% |
| Kraton G1654 | 9.00% | 4.50% | 4.50% | 4.50% | 4.50% |
| Kraton G1641H | | 2.50% | 2.50% | 2.50% | 2.50% |
| Kraton MD6945-10 | | 2.50% | 2.50% | 2.50% | 2.50% |
| Septon 2063 | | 2.00% | 3.00% | 2.00% | 2.00% |
| Septon 4055 alternate Kraton G1651H | 3.00% | 2.50% | 2.50% | 2.50% | 2.50% |
| Septon 4033 alternate Kraton G1650 | 2.00% | 1.00% | 1.00% | 1.00% | 1.00% |
| Eastman Regalrez 1094 | | 1.00% | 2.00% | 1.70% | 1.70% |
| Eastman Foral AX Rosin | | 1.00% | 2.00% | 2.05% | 2.55% |
| Eastotac H100-W Rosin | | 1.50% | 3.00% | 3.50% | 4.00% |
| | 100.00% | 100.00% | 100.000% | 100.00% | 100.00% |

| Component | SCM505 | SCM506 | SCM506R2 | SCM507 | SCM507A |
|---|---|---|---|---|---|
| Pure Syn 2 (Hydrogenated Polydecen) | 48.77% | 49.00% | 49.00% | | |
| Duoprime 70/Clarion 70 Mineral Oil | | | | 19.00% | 19.00% |
| Duoprime 200 Mineral Oil | | | | 38.88% | 38.88% |
| Octyl Palmitate | 6.14% | 6.00% | 5.85% | | |
| Coconut Oil | 4.39% | 4.00% | 4.00% | | |
| MCT Oil 70/30 (Capric/Caprylic | 1.75% | 1.50% | 1.30% | | |
| Triglycerides) | | | | | |
| High Linoleic Safflower Oil | 0.88% | 1.00% | 1.00% | 1.75% | 1.75% |
| Vitamin A Palmitate | 0.88% | 1.00% | 1.00% | 1.00% | 1.00% |
| SymRise SymRepair (TM) 153884 | 0.44% | 0.50% | 0.75% | 0.75% | 0.75% |
| Quercetin Extract | 0.18% | 0.20% | 0.20% | 0.20% | 0.20% |
| Ceramide 3 | 0.14% | 0.15% | | | |
| p-Menthane 3,8 diol (Lipo Coolact 38D) | 0.44% | 0.50% | 0.75% | 0.75% | 0.75% |
| Vitamin E Acetate | 0.44% | 0.50% | 0.50% | 0.50% | 0.50% |
| Propyl Paraben | 0.02% | 0.02% | 0.02% | | |
| BHT | 0.02% | 0.02% | 0.02% | 0.02% | 0.02% |
| Kraton D1117-BT | 8.33% | 10.00% | 10.00% | | |
| Kraton G1654 | 3.95% | 3.00% | 3.00% | | |
| Kraton G1641H | 2.19% | 2.00% | 2.00% | | |
| Kraton MD6945-10 | | 2.00% | 2.00% | | |
| Kraton RP6935 | 3.07% | | | | |
| Septon 2063 | 5.04% | 5.36% | 5.36% | 21.40% | 21.40% |
| Septon 4055 alternate Kraton G1651H | 2.19% | 2.00% | 2.00% | 4.75% | 4.75% |
| Septon 4033 alternate Kraton G1650 | 0.88% | 1.00% | 1.00% | 1.50% | 1.50% |
| Eastman Regalrez 1094 | 3.68% | 4.00% | 4.00% | 2.50% | 2.50% |
| Eastman Foral AX Rosin | 2.24% | 2.25% | 2.25% | 5.00% | 5.00% |
| Eastman Foral 85-E Rosin | | | | | 2.00% |
| Eastotac H100-W Rosin | 3.95% | 4.00% | 4.00% | 2.00% | |
| | 100.00% | 100.00% | 100.00% | 100.00% | 100.00% |

Preparation of the Exemplary Gel Composition Base Formulations

Exemplary gelatinous elastomer composition base formulations are prepared as follows. Oil portions are heated to between 150° C.-175° C. Free fatty acids, triglycerides, and oil soluble biologically active ingredients and botanical or organic extracts are pre-blended with ceramides, vitamins and other ingredients. Liquid portions of formulations are added to copolymers and ester resins in a heated vessel properly equipped to blend the materials homogeneously with minimal entrainment of air. All ingredients are combined and mixed to homogeneity.

### Example 2

### Exemplary Gel Compositions Containing Active Agents

The following are exemplary gel compositions of the present invention that contain one or more active agents. These exemplary gels may be prepared using the preparative methods of the present invention as outlined above, and other methods that are well known in the art for making TPE gel compositions.

06-087CS

| Component | Weight % |
|---|---|
| Puresyn 2 | 51.485% |
| Coconut Oil | 33.500% |
| Kraton 1654 | 9.000% |
| Septon 4055 | 3.000% |
| Septon 4033 | 2.000% |
| Vitamin E | 1.000% |
| BHT | 0.015% |
| | 100.000% |

BS87R1-D

| Component | Weight % |
|---|---|
| Puresyn 2 | 63.500% |
| BS-CT Source Coconut Oil | 18.000% |
| Kraton 1654 | 9.250% |
| Septon 4055 | 3.150% |
| Septon 4033 | 2.100% |
| Vitamin E | 1.000% |
| Sweet Almond Oil | 1.500% |
| BS-CT Source Brazil Nut Oil | 1.500% |
| Soft Almond Spray | 0.150% |
| | 100.150% |

D1027A

| Component | Weight % |
|---|---|
| DuoPrime 200 Mineral Oil | 50.90% |
| Septon 2063 | 28.80% |
| DuoPrime 70 Mineral Oil | 7.50% |
| Septon 4055 | 3.30% |
| Camphor Oil | 3.00% |
| Eastman Regalrez 1094 | 2.00% |
| Foral AX Rosin | 2.00% |
| Septon 4033 | 1.50% |
| L-Menthol Crystals | 1.00% |
| | 100.00% |

SCM003-R1

| Component | Weight % |
|---|---|
| Puresyn 2 | 52.750% |
| Coconut Oil | 13.260% |
| Octyl Palmitate | 7.000% |
| Kraton 1654 | 6.000% |
| High Linoleic Safflower Oil | 5.250% |
| MCT Oil 70/30 | 4.000% |
| Kraton RP6935 | 3.500% |
| Septon S4055 (Alternate Kraton G1651H) | 3.000% |
| Septon S4033 (Alternate Kraton G1650) | 2.000% |
| Vitamin A Palmitate Oil | 1.000% |
| Coolact 38D | 0.750% |
| SymRise SymRepair | 0.750% |
| Vitamin E | 0.500% |
| Quercetin Extract 98% | 0.200% |
| Propyl Paraben | 0.020% |
| BHT | 0.020% |
| | 100.000% |

SCM003-R2

| Component | Weight % |
|---|---|
| Puresyn 2 | 52.750% |
| Coconut Oil | 13.260% |
| Octyl Palmitate | 7.000% |
| Kraton 1654 | 6.000% |
| High Linoleic Safflower Oil | 5.250% |
| MCT Oil 70/30 | 4.000% |
| Kraton RP6935 | 3.500% |
| Septon S4055 (Alternate Kraton G1651H) | 3.000% |
| Septon S4033 (Alternate Kraton G1650) | 2.200% |
| Vitamin A Palmitate Oil | 1.000% |
| Coolact 38D | 0.750% |
| SymRise SymRepair | 0.750% |
| Vitamin E | 0.500% |
| Propyl Paraben | 0.020% |
| BHT | 0.020% |
| | 100.000% |

SCM506-R2

| Component | Weight % |
|---|---|
| Puresyn 2 | 49.000% |
| Kraton D1117P17 (prev. D1117-BT) | 10.000% |
| Octyl Palmitate | 5.850% |
| Septon S2063 | 5.360% |
| Coconut Oil | 4.000% |
| Regalrez 1094 | 4.000% |
| Eastotac H100-W Rosin | 4.000% |
| Kraton 1654 | 3.000% |
| Foral AX Rosin | 2.250% |
| Kraton G1641H | 2.000% |
| Kraton MD6945-10 | 2.000% |
| Septon S4055 (Alternate Kraton G1651H) | 2.000% |
| MCT Oil 70/30 | 1.300% |
| High Linoleic Safflower Oil | 1.000% |
| Vitamin A Palmitate Oil | 1.000% |
| Septon S4033 (Alternate Kraton G1650) | 1.000% |
| SymRise SymRepair | 0.750% |
| Coolact 38D | 0.750% |
| Vitamin E | 0.500% |
| Quercetin Extract 98% | 0.200% |
| Propyl Paraben | 0.020% |
| BHT | 0.020% |
| | 100.000% |

SCM507A

| Component | Weight % |
|---|---|
| DuoPrime 200 Mineral Oil | 38.880% |
| Septon S2063 | 21.400% |
| DuoPrime 70 Mineral Oil | 19.000% |
| Foral AX Rosin | 5.000% |
| Septon S4055 (Alternate Kraton G1651H) | 4.750% |
| Regalrez 1094 | 2.500% |
| Foral 85-E Rosin | 2.000% |
| High Linoleic Safflower Oil | 1.750% |
| Septon S4033 (Alternate Kraton G1650) | 1.500% |
| Vitamin A Palmitate Oil | 1.000% |
| SymRise SymRepair | 0.750% |
| Coolact 38D | 0.750% |
| Vitamin E | 0.500% |
| Quercetin Extract 98% | 0.200% |
| BHT | 0.020% |
| | 100.000% |

SCM507B

| Component | Weight % |
|---|---|
| DuoPrime 200 Mineral Oil | 38.880% |
| Septon S2063 | 21.400% |
| DuoPrime 70 Mineral Oil | 19.000% |
| Foral AX Rosin | 5.000% |
| Septon S4055 (Alternate Kraton G1651H) | 4.750% |
| Regalrez 1094 | 2.500% |
| Foral 85-E Rosin | 2.000% |
| High Linoleic Safflower Oil | 1.750% |
| Septon S4033 (Alternate Kraton G1650) | 1.700% |
| Vitamin A Palmitate Oil | 1.000% |
| SymRise SymRepair | 0.750% |
| Coolact 38D | 0.750% |
| Vitamin E | 0.500% |
| BHT | 0.020% |
| | 100.000% |

D1201A

| Component | Weight % |
|---|---|
| Puresyn 2 | 48.050% |
| Coconut Oil | 27.600% |
| Kraton 1654 | 5.000% |
| Sesame Seed Oil RBWD | 3.000% |
| Wheat Germ Oil | 3.000% |
| Dabur Amla Oil | 3.000% |
| Kraton RP6935 | 2.750% |
| Septon S4055 (Alternate Kraton G1651H) | 2.500% |
| MCT Oil 70/30 | 2.000% |
| Septon S4033 (Alternate Kraton G1650) | 1.500% |
| Vitamin E | 1.000% |
| Croda Hydrolactin 2500 | 0.300% |
| Fragrance Blend Silipos #209-0054 Alpha | 0.240% |
| Methyl Paraben | 0.020% |
| Propyl Paraben | 0.020% |
| BHT | 0.020% |
| | 100.000% |

D1101B

| Component | Weight % |
|---|---|
| Puresyn 2 | 52.350% |
| Coconut Oil | 17.850% |
| Kraton 1654 | 7.900% |
| Kraton D1117P17 (Prev. D1117-BT) | 5.400% |
| Eastotac H100W Resin | 4.400% |
| Avocado Oil | 3.000% |
| Septon 4055 | 2.650% |
| Septon 4033 | 1.800% |
| Foral AX Rosin | 1.730% |
| Vitamin E | 1.000% |
| Padinami | 0.250% |
| Sederma ODA White | 0.250% |
| Fragrance Blend Silipos #209-0054 Alpha | 0.400% |
| Bio Oil Blend | 0.500% |
| SymRise SymRepair | 0.500% |
| BHT | 0.020% |
| | 100.000% |

D1301

| Component | Weight % |
|---|---|
| Puresyn 2 | 60.800% |
| Coconut Oil | 16.380% |
| Kraton 1654 | 6.000% |
| High Linoleic Safflower Oil | 5.200% |
| Kraton RP6935 | 3.500% |
| Septon S4055 | 3.000% |
| Septon S4033 | 2.000% |
| MCT Oil 70/30 | 1.000% |
| SymRise SymRepair | 0.750% |
| Vitamin E | 0.750% |
| Vitamin A Palmitate Oil | 0.300% |
| Sederma ODA White | 0.300% |
| BHT | 0.020% |
| | 100.000% |

D1048ST

| Component | Weight % |
|---|---|
| DP200 | 37.000% |
| Tea Tree Oil | 2.500% |
| Vitamin E | 0.500% |
| Septon 2063 | 30.000% |
| Regalrez 1094 | 30.000% |
| | 100.000% |

### Example 3

### Exudation of Active Agents

Exudation of biologically active agents from exemplary gelatinous elastomer compositions were determined as follows. Gel specimen and Control Sample Oil Formula gels were formulated as follows:

| Control Sample Formula g (w/w %) | | Gel Specimen | |
|---|---|---|---|
| Hydrogenate Polydecene | 4.18 g (51.00%) | Gel formula 06-087CS | 30.0 g (84.77%) |
| Coconut Oil | 2.50 g (30.50%) | Ceramide III | 1.375 g (3.89%) |
| Ceramide III | 0.41 g (5.00%) | Salicylic Acid | 1.375 g (3.89%) |
| Salicylic Acid | 0.41 g (5.00%) | Quercetin | 0.300 g (0.85%) |
| Quercetin | 0.08 g (1.00%) | Methyl Salicylate | 0.780 g (2.20%) |
| Methyl Salicylate | 0.21 g (2.50%) | Vit. A Palmitate | 00.780 g (2.20%) |
| Vit. A Palmitate | 0.21 g (2.50%) | Synthetic Menthol | 0.780 g (2.20%) |
| Synthetic Menthol | 0.21 g (2.50%) | Total: | 35.390 g (100.00%) |
| Total: | 8.20 g (100.00%) | | |

Sample filter paper discs were placed in contact with gel specimens of same diameter under constant low pressure (0.40 psi. (2.8 Mpa)) at 37° C. Multiple time-controlled exposures of the filter paper to the gel were preformed in succession to same side of gel. Quantification of active agent transferred to the filter paper discs was determined by UV, MS, GC and or HPLC analysis after extraction of active agent from the filter paper. Exudation rates was expressed as μg/unit time per unit surface area. The results of tests to measure exudation rates from gelatinous elastomers are set out in Tables 3 and 4.

TABLE 3
Active Agent Transferred from Gel to Filters (μg per filter)

| | | Time of | | | | | | |
|---|---|---|---|---|---|---|---|---|
| Gel | Hours | Filter | | | | | | |
| Specimen | of Filter | Exposure | Salicylic | | Methyl | Vit. A | | Ceramide |
| Number | Exposure | (h) | Acid | Quercetin | Salicylate | Palmitate | S-Menthol | III |
| 1 | 1 | 0-1 | 372 | 10 | 1005 | 841 | 3578 | 18 |
| 2 | 1 | 0-1 | 705 | 12 | 1753 | 1159 | 4855 | 20 |
| 1 | 3 | 1-4 | 793 | 11 | 1694 | 977 | 4193 | 18 |
| 2 | 3 | 1-4 | 1459 | 15 | 2506 | 1284 | 5373 | 24 |
| 1 | 18 | 4-22 | 4765 | 21 | 4812 | 1955 | 8952 | 25 |
| 2 | 18 | 4-22 | 5153 | 30 | 4847 | 1864 | 8916 | 22 |
| 1 | 18 | 22-40 | 6600 | 31 | 5165 | 1682 | 8566 | 29 |
| 2 | 18 | 22-40 | 4976 | 21 | 4271 | 1307 | 7446 | 13 |
| 1 | 18 | 40-58 | 4035 | 15 | 3882 | 916 | 7193 | 10 |
| 2 | 18 | 40-58 | 3941 | 16 | 3729 | 1523 | 7108 | 20 |

TABLE 4
Total Active Agent Exuded from Gel (μg)

| Gel | | | | | | | |
|---|---|---|---|---|---|---|---|
| Specimen | | Salicylic | | Methyl | Vit. A | | Ceramide |
| Number | Time (h) | Acid | Quercetin | Salicylate | Palmitate | S-Menthol | III |
| 1 | 1 | 372 | 10 | 1005 | 841 | 3578 | 18 |
| 2 | 1 | 705 | 12 | 1753 | 1159 | 4855 | 20 |
| 1 | 4 | 1165 | 21 | 2699 | 1818 | 7771 | 35 |
| 2 | 4 | 2164 | 27 | 4259 | 2443 | 10229 | 43 |
| 1 | 22 | 5929 | 42 | 7511 | 3773 | 16723 | 60 |
| 2 | 22 | 7316 | 57 | 9106 | 4307 | 19145 | 65 |
| 1 | 40 | 12529 | 72 | 12675 | 5455 | 25289 | 89 |
| 2 | 40 | 12293 | 78 | 13376 | 5614 | 26590 | 78 |
| 1 | 58 | 16565 | 87 | 16558 | 6370 | 32482 | 99 |
| 2 | 58 | 16234 | 94 | 17106 | 7136 | 33699 | 98 |

The exudation rate from a triglyceride gelatinous elastomer formulation of the invention was more rapid than transfer of active agents from leading mineral oil based gel formulations known in the art. Oil exudation in the first 30 min was 300% greater from the triglyceride gelatinous elastomer formulation, compared to a leading mineral oil based cosmetic gel formula and oil exudation in the first 1 h was 800% from the triglyceride gelatinous elastomer formulation, compared to a leading mineral oil based cushioning gel formula.

### Example 4

### Various Gels with Different Ratios of Mid-Block Solubilizing Oils to Triglyceride Oil and Effect on Weeping, Gel Integrity and Viscosity for Production Molding Purposes

Table 5 provides a qualitative assessment of weeping, gel integrity, and viscosity for eight different gel compositions having different ratios of mid-block solubilizing oils to triglyceride oil.

TABLE 5

| | Compound # | 06087 | T1000 | T1001 | T1002 | T1003 | T1004 | T1008 | T1009 |
|---|---|---|---|---|---|---|---|---|---|
| | Kraton 1654 | 9.00% | | | | 9.00% | | 9.00% | |
| | Septon 4033 | 2.00% | | | | 2.00% | | 2.00% | |
| | Septon 4055 | 3.00% | | | | 3.00% | | 3.00% | |
| | Kraton | | 12% | 14% | 12% | | 14.00% | | 14.00% |
| | RP6935 | | | | | | | | |
| | Coconut Oil | 34.40% | 88% | 36.5% | 29.0% | 28.0% | 28.0% | 35.00% | 35.00% |
| | Palm Oil | | | | | 28.0% | 28.0% | | |
| | Octyl | | | 1.5% | | 1.35% | 1.35% | 0.35% | 0.35% |
| | Palmitate | | | | | | | | |
| | Castor Oil | | | | 30.0% | | | | |
| | Pure Syn 2 | 51.60% | | 34.50% | 29.0% | 20.0% | 20.0% | 35.00% | 35.00% |
| | Solane 6CG | | | 12.85% | | 8.0% | 8.0% | 15.00% | 15.00% |
| | Fragrance | | | 0.15% | 0.15% | 0.15% | 0.15% | 0.15% | 0.15% |
| | (in min oil) | | | | | | | | |
| | Pure Ester | | | 0.50% | | 0.50% | 0.50% | 0.50% | 0.50% |
| | 24 | | | | | | | | |
| | Total | 100.00% | 100.00% | 100.00% | 100.15% | 100.00% | 100.00% | 100.00% | 100.00% |
| | Polymer % | 14.00% | 12.00% | 14.00% | 11.98% | 14.00% | 14.00% | 14.00% | 14.00% |
| | Total Oil % | 86.00% | 88.00% | 86.00% | 88.17% | 86.00% | 86.00% | 86.00% | 86.00% |
| Ratio | Triglyceride/ | 40.00 | 100.0 | 44 | 67 | 67 | 67 | 41 | 41 |
| | non | | | | | | | | |
| | paraffinic | | | | | | | | |
| | Paraffinic | 60.00 | 0.0 | 56 | 33 | 33 | 33 | 59 | 59 |
| | Notes/ | Exudation is | Very | Very High | Clear when | Yellow when | Clear, with | Very | Very |
| | Results | greater than | crystalline | viscosity at | hot, cools off | cool, oily | slight yellow | Promsing, | Promising, |
| | | mineral oil | at RT after 1 | 150 C. (too | to milky white | feeling, | tint initially, | much drier | softer |
| | | only | day. Weak, | thick to | “very exuding” | slightly cloudy | after 3 days, | feeling than | durometer |
| | | formulation. | Waxy, very | pour, lower | weak structure | as cooling | slight | std. | than T1008, |
| | | Viscosity | greasy, no | at 165 C., | gel, “greasy” | progressed, | crystalliztaion | production | very optically |
| | | iscorrect for | tear resistance, | not thin | feeling, slimy | After 3 days, | of oil at | formula, may | clear, does |
| | | molding | low tensile | enough to | | oil | exposed | be not | not feel quite |
| | | operations, | modulus | dip until | | crystallized, | surfaces, less | exuding | as dry to the |
| | | No free oil | | 168-170 C. | | very greasy | greasy than | enough | touch but |
| | | weeping | | | | | T1003 but still | | viscosity is |
| | | | | | | | not acceptable | | quite high |

| | | | | | | | | |
|---|---|---|---|---|---|---|---|---|
| Weeping Index | 1 | 9 | 2 | 9 | 10 | 4 | 1 | 2 |
| (1 = low 10 = high) | | | | | | | | |

To accomplish the above trials, a temperature controlled heating mantle with high speed mixer was employed and 300 grams total for each formulation based on the ratios of ingredients in Table X were prepared. Ingredients were weight out utilizing a digital scale to an accuracy of 0.00 gram and then added in sequence starting by first heating and stirring the oils until they reached the desired temperature of 165° C., then adding the polymers, then mixing at a rate of about 250 RPM using a lab mixer and standard mixing blade for a period of 45 to 60 minutes under heat. Formulas were mixed until smooth and all polymer components were melted and homogeneous within the gel. In the case of formula T1001 above, temperature was adjusted from 150° C. up to 170° C. Adjustment of the temperature above this point was avoided due to being above the flash point of the oils employed. As such, there are upper limits to temperature during process and viscosity at temperatures in the range of about 130° C. to 165 or 170° C. are an important consideration to the formulation for conversion purposes.

### Example 5

### Measuring the Exudation Rates of Active Agents

A gel was prepared by combining Triglyceride gel formulation 06-087CS in the lab with each of the following ingredients: Salicylic Acid, Quercetin, Methyl Salicylate, Vit. A Palmitate, and Synthetic Menthol (menthanediol). The gel with bio-available ingredients was prepared by first heating a 30 gram sample of 06-087CS gel, then by adding the amounts of each of the aforementioned ingredients listed in the table below to the gel and mixing on a heated stirring plate in a beaker.

| Gel Disc Formula | | g | % w/w |
|---|---|---|---|
| | 06-087CS Gel | 30.000 | 84.77% |
| Plus | Ceramide III | 1.375 | 3.89% |
| | Salicylic Acid | 1.375 | 3.89% |
| | Quercetin | 0.300 | 0.85% |
| | Methyl Salicylate | 0.780 | 2.20% |
| | Vit. A Palmitate | 0.780 | 2.20% |
| | Synthetic Menthol | 0.780 | 2.20% |
| | | 35.390 | 100.00% |

This compound was then divided into three aliquots' of approximately 10 grams each and poured into a 2.625″ diameter aluminum petri dish and allowed to cool to create individual gel samle discs. Subsequently, absorbent cellulose filter blotter paper was die cut to enough 2.625″ diameter discs to be employed as a wicking media for repeated exposure in direct contact with the gel compound under specific conditions as follows.

Exposure Conditions: 37-40 deg. C., Sample filter paper discs in contact with gel specimens of same diameter were stacked in 50 mm dia. aluminum petri dishes, gel specimen diameter 2.625″×0.125″ thick, filter paper disc diameter 2.625″ with another petri dish and a 1 kg dead weight on top of stack. Pressure calculated at ˜0.407 psi. (2.8 Mpa) constant. Two replicates per test condition. Repeat exposures at 3 hours then repeat three exposures of 18 hours each, changing discs but keeping same gel specimen and same side of each gel specimen in contact with each successive filter paper disc sample.

The control sample oil and ingredient only (no polymer as contained in the 06-087CS gel, but the same ratio of oils and other ingredients) mixture formula was prepared as follows:

| Control Sample Oil Formula | grams | w/w % |
|---|---|---|
| Hydrogenated Polydecene | 4.18 | 51.00% |
| Coconut Oil | 2.50 | 30.50% |
| Ceramide III | 0.41 | 5.00% |
| Salicylic Acid | 0.41 | 5.00% |
| Quercetin | 0.08 | 1.00% |
| Methyl Salicylate | 0.21 | 2.50% |
| Vit. A Palmitate | 0.21 | 2.50% |
| Synthetic Menthol | 0.21 | 2.50% |
| | 8.20 | 100.00% |

Two Control filter paper disc samples were then soaked in control sample formula oil and exposed to the same conditions as the test samples served to determine % volatilized of each ingredient during mixing and test condition exposure and subsequent shipping to an analytical testing lab for HPLC and GC testing.

Data was calculated to determine the total weight of exudate absorbed by each individual sample filter paper blotter disc test specimen. Calculations were then performed based upon the ratios of ingredients in the gel disc formula to determine the theoretical amount of each ingredient that could have been exuded from the oil if the oil contained the same ratio of ingredients as contained in the control oil sample and in the gel. That data is detailed below:

| | | | | | | Estimated | | Estimated | | Estimated | Control Disc |
|---|---|---|---|---|---|---|---|---|---|---|---|
| | | | Wt. | | Oil | grams | Estimated | Grams | Estimated | grams | [C1/C2] Wt. |
| control | Sample | Sample | Before | Wt. After | Exuded - | Salicylic | grams | Methyl | grams Vit. A | Synthetic | after |
| ID | I.D. | Description | Exposure | Exposure | Absorbed | Acid | Quercetin | Salicylate | Palmitate | Menthol | Exposure |
| c1 | aa | Test Disc 1, | 1.15 | 1.25 | 0.10 | 0.0039 | 0.0009 | 0.0022 | 0.0022 | 0.0022 | 2.74 |
| | | Exposed 1 hours | | | | | | | | | |
| c2 | bb | Test Disc 1 | 1.14 | 1.28 | 0.14 | 0.0054 | 0.0012 | 0.0031 | 0.0031 | 0.0031 | 2.12 |
| | | Replicate, | | | | | | | | | |
| | | Exposed 1 hours | | | | | | | | | |
| c1 | cc | Test Disc 2, | 1.12 | 1.23 | 0.11 | 0.0043 | 0.0009 | 0.0024 | 0.0024 | 0.0024 | 2.74 |
| | | Exposed 3 hours | | | | | | | | | |
| c2 | dd | Test Disc 2 | 1.06 | 1.22 | 0.16 | 0.0062 | 0.0014 | 0.0035 | 0.0035 | 0.0035 | 2.12 |
| | | Replicate, | | | | | | | | | |
| | | Exposed 3 hours | | | | | | | | | |
| c1 | ee | Test Disc 3, | 1.14 | 1.42 | 0.28 | 0.0109 | 0.0024 | 0.0062 | 0.0062 | 0.0062 | 2.73 |
| | | Exposed 18 hours | | | | | | | | | |
| c2 | ff | Test Disc 3 | 1.09 | 1.37 | 0.28 | 0.0109 | 0.0024 | 0.0062 | 0.0062 | 0.0062 | 2.11 |
| | | Replicate, | | | | | | | | | |
| | | Exposed 18 hours | | | | | | | | | |
| c1 | gg | Test Disc 4, | 1.10 | 1.39 | 0.29 | 0.0113 | 0.0025 | 0.0064 | 0.0064 | 0.0064 | 2.72 |
| | | Exposed 18 hours | | | | | | | | | |
| c2 | hh | Test Disc 4 | 1.11 | 1.37 | 0.26 | 0.0101 | 0.0022 | 0.0057 | 0.0057 | 0.0057 | 2.10 |
| | | Replicate, | | | | | | | | | |
| | | Exposed 18 hours | | | | | | | | | |
| c1 | ii | Test Disc 5, | 1.09 | 1.32 | 0.23 | 0.0089 | 0.0020 | 0.0051 | 0.0051 | 0.0051 | 2.72 |
| | | Exposed 18 hours | | | | | | | | | |
| c2 | kk | Test Disc 5 | 1.13 | 1.36 | 0.23 | 0.0089 | 0.0020 | 0.0051 | 0.0051 | 0.0051 | 2.1 |
| | | Replicate, | | | | | | | | | |
| | | Exposed 18 hours | | | | | | | | | |

The filter paper disc specimens aa through kk were then sent to an analytical laboratory for HPLC and GC analysis along with a small sample of each individual raw ingredient so that the HPLC and GC equipment could be calibrated for the protocol being employed by the lab. This calibration allowed for determining the maximum recoverable (measurable) amount of each ingredient that could be measured using these techniques and would later be used to correct the data. The analytical laboratory then detected and measured the amount of each of the added active agents that was flushed out of the filter paper discs using wet chemistry techniques known to those skilled in the art.

The analytical data was interpreted, and the amounts of each ingredient which was exuded into each filter paper blotter disc specimen, and subsequently extracted using wet chemistry, were then entered into a data analysis spreadsheet and adjusted based on the Analytical Recovery Rate to determine actual concentration of each ingredient extracted.

The results of those calculations are below:

| | | | Quantitative | | | | | | Analytical Recovery Rate, | | | | | |
|---|---|---|---|---|---|---|---|---|---|---|---|---|---|---|
| | | | Analysis - Islechem | | | | | | Adjusted Concentration (ug) | | | | | |
| | | | u-gram extraction | | | | | | 0.85 | | | | | |
| | | | Sali- | | Methyl | | | | Sali- | 0.89 | 0.85 | 0.88 | 0.83 | 0.97 |
| | Cummulative | Spec- | cylic | Quer- | Sali- | Vit. A | S- | Ceramide | cylic | Quer- | Methyl | Vit. A | S- | Ceramide |
| Hours | Hours | imen | Acid | cetin | cylate | Palmitate | Menthol | III | Acid | cetin | Salicylate | Palmitate | Menthol | III |
| 1 | 1 | aa | 316 | 9 | 854 | 740 | 2970 | 17 | 372 | 10 | 1005 | 841 | 3578 | 18 |
| 1 | 1 | bb | 599 | 11 | 1490 | 1020 | 4030 | 19 | 705 | 12 | 1753 | 1159 | 4855 | 20 |
| 3 | 4 | cc | 674 | 9.4 | 1440 | 860 | 3480 | 17 | 793 | 11 | 1694 | 977 | 4193 | 18 |
| 3 | 4 | dd | 1240 | 13.2 | 2130 | 1130 | 4460 | 23 | 1459 | 15 | 2506 | 1284 | 5373 | 24 |
| 18 | 22 | ee | 4050 | 18.6 | 4090 | 1720 | 7430 | 24 | 4765 | 21 | 4812 | 1955 | 8952 | 25 |
| 18 | 22 | ff | 4380 | 28.6 | 4120 | 1640 | 7400 | 21 | 5153 | 30 | 4847 | 1864 | 8916 | 22 |
| 18 | 40 | gg | 5610 | 27.5 | 4390 | 1480 | 7110 | 28 | 6600 | 31 | 5165 | 1682 | 8566 | 29 |
| 18 | 40 | hh | 4230 | 19 | 3630 | 1150 | 6180 | 13 | 4976 | 21 | 4271 | 1307 | 7446 | 13 |
| 18 | 58 | ii | 3430 | 13.2 | 3300 | 806 | 5970 | 9.6 | 4035 | 15 | 3882 | 916 | 7193 | 10 |
| 18 | 58 | kk | 3350 | 14.2 | 3170 | 1340 | 5900 | 19 | 3941 | 16 | 3729 | 1523 | 7108 | 20 |

These measurements can then be graphed as per unit time or cumulative per unit time. These results can then be graphed as amount exuded per unit time, then divided by square area of the filter paper in order to obtain a standardized measurement of amount of exudation of each ingredient per unit time per unit area in terms of ug/hour/sq. cm. This unit measurement is a key parameter for employment of gelatinous elastomer compounds as an ingredient delivery system to the body of a mammal for purposes of calculating maximum available dosage which could be delivered to the body and or subsequently absorbed by the body. It is within the ordinary skill in the art to use this method and similar methods of exposing a gelatinous elastomer compound containing bio-available ingredients to an absorbent media (in this case, the filter blotter paper) under specified conditions in order to obtain a measure of exudation rate of these ingredients to the body.

### Example 6

### Adjustment of Exudation Rate Via Adjusting Triglyceride Oil to Mid-Block Solubilizing Oil to Ratio and Polymer Content

The following tables show how the exudation rate can be adjusted by adjusting the triglyceride oil to mid-block solubilizing oil ratio (T:M Ratio) and the polymer content. These studies were conducted at 37° C. A graphical representation of the results of these experiments is shown in FIG. 3.

| | | | Cummulative | Exudation in |
|---|---|---|---|---|
| Gel | Elapsed | Weight | Exudation | mg per cm\hat{ }2 |
| Formulation | Minutes | (g) | (g) | of gel |
| S22B - 1:0 | 0 | 4.55 | 0 | 0.00 |
| T:M Ratio, | 30 | 4.60 | 0.05 | 2.47 |
| 14% Polymer | 60 | 4.63 | 0.08 | 3.95 |
| | 90 | 4.66 | 0.11 | 5.43 |
| | 120 | 4.68 | 0.13 | 6.42 |
| | 150 | 4.70 | 0.15 | 7.41 |
| | 180 | 4.715 | 0.165 | 8.15 |
| | 210 | 4.73 | 0.18 | 8.89 |
| | 240 | 4.74 | 0.19 | 9.38 |
| | 270 | 4.75 | 0.20 | 9.88 |
| | 300 | 4.79 | 0.24 | 11.85 |
| | 360 | 4.81 | 0.26 | 12.84 |
| | 420 | 4.85 | 0.30 | 14.81 |
| | 480 | 4.87 | 0.32 | 15.80 |
| | 1440 | 5.10 | 0.55 | 27.16 |
| 06-087B - 0.667:1 | 0 | 4.60 | 0 | 0.00 |
| T:M Ratio, | 30 | 4.66 | 0.06 | 2.96 |
| 14% Polymer | 60 | 4.68 | 0.08 | 3.95 |
| | 90 | 4.70 | 0.10 | 4.94 |
| | 120 | 4.72 | 0.12 | 5.93 |
| | 150 | 4.73 | 0.13 | 6.42 |
| | 180 | 4.745 | 0.145 | 7.16 |
| | 210 | 4.76 | 0.16 | 7.90 |
| | 240 | 4.77 | 0.17 | 8.40 |
| | 270 | 4.80 | 0.20 | 9.88 |
| | 300 | 4.81 | 0.21 | 10.37 |
| | 360 | 4.83 | 0.23 | 11.36 |
| | 420 | 4.85 | 0.25 | 12.35 |
| | 480 | 4.865 | 0.265 | 13.09 |
| | 1440 | 5.00 | 0.40 | 19.75 |
| 121096 - 0:1 | 0 | 4.52 | 0 | 0.00 |
| T:M Ratio, | 30 | 4.52 | 0.00 | 0.00 |
| 12.6% Polymer | 60 | 4.53 | 0.01 | 0.49 |
| | 90 | 4.53 | 0.01 | 0.49 |
| | 120 | 4.53 | 0.01 | 0.49 |
| | 150 | 4.53 | 0.01 | 0.49 |
| | 180 | 4.535 | 0.015 | 0.74 |
| | 210 | 4.54 | 0.020 | 0.99 |
| | 240 | 4.54 | 0.02 | 0.99 |
| | 270 | 4.545 | 0.025 | 1.23 |
| | 300 | 4.545 | 0.03 | 1.23 |
| | 360 | 4.55 | 0.03 | 1.48 |
| | 420 | 4.55 | 0.03 | 1.48 |
| | 480 | 4.55 | 0.03 | 1.48 |
| | 1440 | 4.55 | 0.03 | 1.48 |
| D1028B - Ratio 4:1 | 0 | 4.67 | 0 | 0.00 |
| T:M Ratio, | 30 | 4.69 | 0.02 | 0.99 |
| 16.8% Polymer | 60 | 4.71 | 0.04 | 1.98 |
| | 90 | 4.72 | 0.05 | 2.47 |
| | 120 | 4.73 | 0.06 | 2.96 |
| | 150 | 4.74 | 0.07 | 3.46 |
| | 180 | 4.76 | 0.09 | 4.44 |
| | 210 | 4.78 | 0.11 | 5.43 |
| | 240 | 4.79 | 0.12 | 5.93 |
| | 270 | 4.80 | 0.13 | 6.42 |
| | 300 | 4.81 | 0.14 | 6.91 |
| | 360 | 4.82 | 0.15 | 7.41 |
| | 420 | 4.83 | 0.16 | 7.90 |
| | 480 | 4.84 | 0.17 | 8.40 |
| | 1440 | 4.91 | 0.24 | 11.85 |
| 71498 - Ratio 0.06:1 | 0 | 4.55 | 0 | 0.00 |
| T:M Ratio, | 30 | 4.57 | 0.02 | 0.99 |
| 12.6% Polymer | 60 | 4.58 | 0.03 | 1.48 |
| | 90 | 4.59 | 0.04 | 1.98 |
| | 120 | 4.59 | 0.04 | 1.98 |
| | 150 | 4.60 | 0.05 | 2.47 |
| | 180 | 4.605 | 0.055 | 2.72 |
| | 210 | 4.61 | 0.06 | 2.96 |
| | 240 | 4.63 | 0.08 | 3.95 |
| | 270 | 4.635 | 0.085 | 4.20 |
| | 300 | 4.64 | 0.09 | 4.44 |
| | 360 | 4.65 | 0.10 | 4.94 |
| | 420 | 4.66 | 0.11 | 5.43 |
| | 480 | 4.66 | 0.11 | 5.43 |
| | 1440 | 4.68 | 0.13 | 6.42 |

Having fully described the invention, it will be appreciated by those skilled in the art that the invention can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is thus intended to cover, for example and without limitation, any variations, uses, or adaptations of the inventions following, in general, the principles of the invention and including such departures from the disclosure that as come within known or customary practice within the art to which the invention pertains and as may be applied to the essential features hereinbefore set forth.

What is claimed:

1. A gelatinous elastomer composition comprising from about 1.0% to about 50.0% by weight of a block copolymer, from about 1% to 99% by weight of a mid-block solubilizing oil and from about 1% to 99% by weight of a triglyceride oil, wherein
the ratio of the triglyceride oil to the mid-block solubilizing oil is between about 1:100 to 3:1.

2. The gelatinous elastomer composition of claim 1, wherein the mid-block solubilizing oil is a mineral or a synthetic oil.

3. The gelatinous elastomer composition of claim 2, wherein the ratio of the triglyceride oil to the mid-block solubilizing oil is between about 1:100 and about 50:50.

4. The gelatinous elastomer composition of claim 3, comprising from about 10% to 90% of the mid-block solubilizing oil by weight, and from about 10% to about 90% of the triglyceride oil by weight.

5. The gelatinous elastomer composition of claim 4, wherein the ratio of the triglyceride oil to the mid-block solubilizing oil is between about 7:70 and about 50:50.

6. The gelatinous elastomer composition of claim 4, wherein the ratio of the triglyceride oil to the mid-block solubilizing oil is between about 15:60 and about 50:50.

7. The gelatinous elastomer composition of claim 2, comprising between about 4% and about 25% by weight of the block copolymer, between about 10% and about 70% by weight of the triglyceride oil, and between about 40% and about 60% by weight of the mineral or synthetic oil.

8. The gelatinous elastomer composition of claim 2, comprising between about 10% and about 25% by weight by weight of the block copolymer, between about 20% and about 60% by weight of the triglyceride oil, and between about 30% and about 70% by weight of the mineral or synthetic oil.

9. The gelatinous elastomer composition of claim 1, wherein the ratio of the triglyceride oil to the mid-block solubilizing oil is between about 1:2 and about 2:1.

10. The gelatinous elastomer composition of claim 1, wherein the ratio of the triglyceride oil to the mid-block solubilizing oil is between about 40:60 and about 60:40.

11. The gelatinous elastomer composition of claim 3, further comprising up to about 15.0% of one or more free fatty acids.

12. The gelatinous elastomer composition of claim 3, further comprising one or more biologically active agents

13. The gelatinous elastomer composition of claim 3, wherein the one or more biologically active agents are selected from the group consisting of Allantoin, Aloe Vera Oil, Alpha-Hydroxy Acid, Aluminum Hydroxide, Aspirin, Bacitracin, Benzoic Acid, Benzalkonium Chloride, Benzocaine, Beta-Hydroxy Acid, BHA, BHT, Bio Oil, Bisabolol, Bleomycim, Benzoic Acid, Boric Acid, Calcium Undecylenate, Calamine, Collagen, Camphor, Capric Acid, Caprylic Acid, Centella Asiatica, Ceramide 2, Ceramide 3, Ceramide 6, Chloral Hydrate, Clioquinol, Colloidal Oatmeal, Corticosteroids, Cyclomethicane Sulfate, Elderflower Extract, Emu Oil, Eugenol, Fouorouracil, Free Fatty Acids, Ferric Chloride, Ginkgo Biloba, Glycerin, Glycol Salicylate, Glycolic Acid, Glycosaminoglycans, Gotu kola, Grape Seed Extract, Helix Aspersa Muller Glycoprotein, Hexyresorcinol, Histamine dihydrochloride, Hyaluronic Acid, Hydrogen Peroxide, Imiquimod, Interferons, Linoleic Acid, Menthol, Menthoxypropanediol, Methyl Salicylate, Methylparaben, Miconasole Nitrate, Neomycin Sulfate, Oleic Acid, Oxyquinoline Sulfate, Panthenol, Penacycline triterpene resin, Phenol, Phenyl Salicylate, Povidone-vinylacetate copolymers, Propionic Acid, Propylparaben, Protein Hydrolysate, Purcellin Oil, Pyridoxine Hydrochloride, Quercetin, Resorcinol, Retinoic Acid, Retinol, Safflower oil, Salicylamide, Salicylic Acid, Silver Nitrate, Silver Ion, Simethicone, Sodium, Propionate, Sodium Salicylate, Sulfur, Tamanu Oil, Tamoxifin, Tannic Acid, Tea tree oil, Tetracycline Hydrochloride, Thymol, Tolindate, Tolnaftate, Topical Starch, Transforming Growth Factors, Trolamine, Trolamine Salicylate, Undecylenic Acid, Vitamin A Palmitate, Vitamin C, Vitamin D, Vitamin E Acetate, Zinc Acetate, Zinc Carbonate, Zinc Chloride, Zinc Oxide, Zinc Propionate, Zinc Sulfate, p-Menthane 3,8 diol Methanediol, Octadecenadioic Acid, Glyceryl Hydrogenated Rosinate, Hydrogenated Gum Rosin, Pentaaerythrityl Hydrogenated Rosinate, Padinami Extract, Natural or Synthetic Ceramides (e.g., Ceramide BIO391, Synthetic Ceramides), Stearic Acid, Phytosterol, Lidocaine Hydrochloride.

14. The gelatinous elastomer composition of claim 13, comprising from about 0% to about 20% by weight of the at least one biologically active agent.

15. The gelatinous elastomer composition of claim 3, wherein the triglyceride oil is selected from the group consisting of Capric Triglyceride, Caprylic Triglyceride, Hydrogenated Vegetable Oil, A Persea Gratissima (Avocado) Oil, Prunus Amygdalys Dulcis (Sweet Almond) Oil, Vitis Vinifera (Grape Seed) Oil, Glycine Soja (Soybean) Oil, Simmonsia Chinensis (Jojoba) Seed Oil, Prunus Armeniaca (Apricot Kernel) Oil, Clear Simmonsia Chinensis (Jojoba) Seed Oil, Sesamum Indicum (Sesame) Oil, Carthamus Tinctorius (Hybrid Safflower) Oil, Carthamus Tinctorius (Safflower) Oil, Juglans Regia (Walnut) Oil, Trictum Vulgare (Wheat Germ) Oil, Hellanthus Annuus (Sunflower Seed) Oil, Fractionated Coconut Oil, Guineenis (Palm) Oil, Olea Europaea (Olive) Oil, (Pale Pressed) Ricinus Communis (Castor) Oil, Macadamia Ternifolia Nut Oil, Hydrogenated Soybean Oil, Canola Oil, Rosa Canina Fruit Oil, Lite Rosa Canina Fruit Oil, Corylus Americana (Hazelnut) Oil, Oryza Sativa (Rice Bran) Oil, Balsam Copaiba, Brassica Campestris (Rapeseed) Oil, Rubus Idaeus (Raspberry) Seed Oil, Oleic/Palmitoleic/Linoleic Glycerides, Hydrogenated Avocado Oil, Andiloba Oil, Aloe Vera Oil, Corn Oil, Wheat Oil, Palm Kernel Oil, Brazil Nut Oil, Peanut Oil, Refined Sunflower Seed Oil and other Hydrogenated or non-hydrogenated processed and refined Vegetable, Fruit Seed and Plant Oils and fractionated derivatives thereof.

16. The gelatinous elastomer composition of claim 1, wherein the block copolymer comprises a styrene derived end block.

17. The gelatinous elastomer composition of claim 3, wherein the block copolymer is a styrene-ethylene/butylene-styrene copolymer, a styrene-ethylene/propylene-styrene copolymer, a hydrogenated styrene-isoprene/butadiene copolymer, a hydrogenated styrene-isoprene copolymer, a hydrogenated styrene-ethylene/butylene-styrene copolymer, a styrene isoprene/butadiene copolymer, a hydrogenated styrene isoprene block copolymer, or a hydrogenated styrene-ethylene/butylene-styrene copolymer.

18. The gelatinous elastomer composition of claim 3, wherein the mid-block solubilizing oil is a mineral oil, a hydrogenated polydecene, a polyisobutene, a hydrogenated didecene, tridecyl stearate, neopentyl glycol dicaprylate, neopentyl glycol dicaprate, tridecyl trimellitate, tridecyl stearate, dipentaerythrityl hexacaprylate, dipentaerythrityl hexacaprate, or octyl palmitate, or mixtures thereof.

19. A molded article comprising the gelatinous elastomer composition of claim 1.

20. A molded article comprising the gelatinous elastomer composition of claim 3.

21. The molded article of claim 20, wherein the article is selected from the group consisting of a, glove, sock, bootie, cuff, sleeve, band, belt, pad, cylinder, patch, leggings, pants, undergarment, or internal body cavity devices designed to deliver portions of said gelatinous elastomer composition to a skin, body tissue or hair.

22. The molded article of claim 21, wherein the article is a glove or a sock.

23. A flexible article comprising a textile fabric coated on at least one side with the gelatinous elastomer composition of claim 1.

24. The flexible article of claim 23, wherein the fabric comprises one or more fibers selected from as polyester, polyamide, polyolefin, acrylic cotton, cambric, wool, cashmere, rayon, and jute.

25. A method for providing a gelatinous elastomer composition having a desired rate of biologically active agent delivery to the human or animal body, the method comprising:
a) providing a gelatinous elastomer composition of claim 12, said composition having a ratio of a triglyceride oil to a mid-block solubilizing oil;
b) contacting the gelatinous elastomer composition with a material capable of absorbing the biologically active agent;
c) measuring the rate at which the biologically active agent is absorbed onto the material;
d) correlating the absorption rate with the delivery rate to the human or animal body;
e) providing an additional gelatinous elastomer composition of claim 12, wherein
if the absorption rate at which the biologically active agent present in the gelatinous elastomer composition of step (a) is lower than the desired delivery rate, the ratio of the triglyceride oil to the mid-block solubilizing oil is increased and
if the absorption rate at which the biologically active agent present in the gelatinous elastomer composition of step (a) is higher than the desired delivery rate, the ratio of the triglyceride oil to the mid-block solubilizing oil is decreased;
f) repeating steps (b)-(d) with the additional gelatinous elastomer composition;
g) providing yet an additional gelatinous elastomer composition of claim 12, wherein
if the absorption rate at which the biologically active agent present in the gelatinous elastomer composition of the prior additional gelatinous elastomer composition is lower than the desired delivery rate, the ratio of the triglyceride oil to the mid-block solubilizing oil is increased and
if the absorption rate at which the biologically active agent present in the gelatinous elastomer composition of the prior additional gelatinous elastomer composition is higher than the desired delivery rate, the ratio of the triglyceride oil to the mid-block solubilizing oil is decreased;
h) repeating steps (f) and (g) as many times as are necessary until a gelatinous elastomer composition having the desired rate of delivery is provided.

26. A method for reducing the discoloration and thickness of keloid and hypertrophic scars, comprising the steps of:
a) providing a substrate and providing the gelatinous elastomer composition of claim 3, wherein the gelatinous elastomer composition comprises one or more of a coconut oil, capric triglycerides, caprylic triglycerides, free fatty acids, high linoleic acid natural oils (for example safflower oil), high oleic acid natural oils, grape seed oil, avocado oil, jojoba oil, canola oil, ceramides, bisabolol, hexyldecanol, Cetylhydroxyproline Palmitamide, Stearic Acid and Brassica Campestris (Rapeseed) Sterols, Padina Pavonica Thallus Extract, aloe, p-menthane 3,8 diol and mixtures thereof;
b) optionally incorporating a therapeutically active agent selected from the group consisting of Vitamins A, B₁₂, C, D, E, and mixtures thereof into the gelatinous elastomer composition,
c) bonding the gelatinous elastomer composition to the substrate;
d) forming the substrate having the gelatinous elastomer composition bonded thereto into a molded article; and
e) wearing the molded article on the keloid or hypertrophic scar for an extended period of time.

27. The gelatinous elastomer composition of claim 12, wherein the biologically active agent is one or more fungicide agents selected aliphatic nitrogen fungicides: butylamine, cymoxanil, dodicin, dodine, guazatine, iminoctadine amide fungicides: carpropamid, chloraniformethan, cyflufenamid, diclocymet, ethaboxam, fenoxanil, flumetover, furametpyr, isopyrazam, mandipropamid, penthiopyrad. Prochloraz, quinazamid, silthiofam, triforine acylamino acid fungicides; benalaxyl, benalaxyl-M, furalaxyl, metalaxyl, metalaxyl-M, pefurazoate, valifenalate, anilide fungicides: benalaxyl, benalaxyl-M, bixafen, boscalid, carboxin, fenhexamid, isotianil, metalaxyl, metalaxyl-M, metsulfovax, ofurace, oxadixyl, oxycarboxin, penflufen, pyracarbolid, sedaxane, thifluzamide, tiadinil, benzanilide fungicides: benodanil, flutolanil, mebenil, mepronil, salicylanilide, tecloftalam, furanilide fungicides, fenfuram, furalaxyl, furcarbanil, methfuroxam, sulfonanilide fungicides, flusulfamide, benzamide fungicides: benzohydroxamic acid, fluopicolide, fluopyram, tioxymid trichlamide, zarilamid, zoxamide, furamide fungicides, cyclafuramid, furmecyclox, phenylsulfamide fungicides: dichlofluanid, tolylfluanid, sulfonamide fungicides amisulbrom, cyazofamid, valinamide fungicides: benthiavalicarb, iprovalicarb, antibiotic fungicides, aureofungin, blasticidin-S, cycloheximide, griseofulvin, kasugamycin, natamycin, polyoxins, polyoxorim, streptomycin, validamycin, strobilurin fungicides, azoxystrobin, dimoxystrobin, fluoxastrobin, kresoxim-methyl, metominostrobin, orysastrobin picoxystrobin, pyraclostrobin, pyrametostrobin, pyraoxystrobin, trifloxystrobin, aromatic fungicides: biphenyl, chlorodinitronaphthalene, chloroneb, chlorothalonil, cresol, dicloran, hexachlorobenzene, pentachlorophenol, quintozene, sodium pentachlorophenoxide, tecnazene, benzimidazole fungicides: benomyl, carbendazim, chlorfenazole, cypendazole, debacarb, fuberidazole, mecarbinzid, rabenzazole, thiabendazole, benzimidazole precursor fungicides: furophanate, thiophanate, thiophanate-methyl, benzothiazole fungicides: bentaluron, benthiavalicarb, chlobenthiazone, probenazole, TCMTB, bridged diphenyl fungicides: bithionol, dichlorophen, diphenylamine, carbamate fungicides: benthiavalicarb, furophanate, iprovalicarb, propamocarb, pyribencarb, thiophanate, thiophanate-methyl, benzimidazolylcarbamate fungicides: benomyl, carbendazim, cypendazole, debacarb, mecarbinzid, carbanilate fungicides: diethofencarb, pyraclostrobin, pyrametostrobin, conazole fungicides: climbazole, clotrimazole, imazalil, ketoconazole, oxpoconazole, prochloraz, triflumizole, azaconazole, bromuconazole, cyproconazole, diclobutrazol, difenoconazole, diniconazole, diniconazole-M, epoxiconazole, etaconazole fenbuconazole, fluquinconazole, flusilazole, flutriafol, furconazole, furconazole-cis, hexaconazole, imibenconazole, ipconazole, miconazole nitrate, metconazole myclobutanil, penconazole, propiconazole, prothioconazole, quinconazole, simeconazole, tebuconazole, tetraconazole, triadimefon, triadimenol, triticonazole, uniconazole uniconazole-P, copper fungicides: Bordeaux mixture, Burgundy mixture, Cheshunt mixture, copper acetate, copper carbonate, basic copper hydroxide, copper naphthenate, copper oleate, copper oxychloride, copper silicate, copper sulfate, copper sulfate, basic copper zinc chromate, cufranebm, cuprobam, cuprous oxide, mancopper, oxine-copper, dicarboximide fungicides: famoxadone, fluoroimide, dichlorophenyl dicarboximide, fungicides: chlozolinate, dichlozoline, iprodione isovaledione, myclozolin, procymidone, vinclozolin, phthalimide fungicides: captafol, captan, ditalimfos, folpet, thiochlorfenphim, dinitrophenol fungicides: binapacryl, dinobuton, dinocap, dinocap-4, dinocap-6, meptyldinocap, dinocton, dinopenton, dinosulfon, dinoterbon, DNOC, dithiocarbamate fungicides: azithiram, carbamorph, cufraneb, cuprobam, disulfiram, ferbam, metam, nabam, tecoram, thiram, ziram, cyclic dithiocarbamate fungicides: dazomet, etem, milneb, polymeric dithiocarbamate fungicides: mancopper, mancozeb, maneb, metiram, polycarbamate, propineb, zineb, imidazole fungicides: cyazofamid, fenamidone, fenapanil, glyodin, iprodione, isovaledione, pefurazoate, triazoxide, inorganic fungicides: potassium azide, potassium thiocyanate, sodium azide, sulfur, inorganic mercury fungicides: mercuric chloride, mercuric oxide, mercurous chloride, organomercury fungicides: (3-ethoxypropyl)mercury bromide, ethylmercury acetate, ethylmercury bromide, ethylmercury chloride, ethylmercury 2,3-dihydroxypropyl, mercaptide, ethylmercury phosphate, N-(ethylmercury)-p-toluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriurea, phenylmercury acetate, phenylmercury chloride, phenylmercury derivative of pyrocatechol, phenylmercury nitrate phenylmercury salicylate, thiomersal, tolylmercury acetate, morpholine fungicides: aldimorph, benzamorf, carbamorph, dimethomorph, dodemorph, fenpropimorph, flumorph, tridemorph, organophosphorus fungicides: ampropylfos, ditalimfos, edifenphos, fosetyl, hexylthiofos, iprobenfos, phosdiphen, pyrazophos, tolclofos-methyl, triamiphos, organotin fungicides: decafentin, fentin, tributyltin oxide, oxathiin fungicides: carboxin, oxycarboxin, oxazole fungicides: chlozolinate, dichlozoline, drazoxolon, famoxadone, hymexazol, metazoxolon, myclozolin, oxadixyl, vinclozolin, polysulfide fungicides: barium polysulfide, calcium polysulfide, potassium polysulfide, sodium polysulfide, pyrazole fungicides: bixafen, furametpyr, isopyrazam, penflufen, penthiopyrad, pyraclostrobin, pyrametostrobin, pyraoxystrobin, rabenzazole, sedaxane, pyridine fungicides: boscalid, buthiobate, dipyrithione, fluazinam, fluopicolide, fluopyram, pyribencarb, pyridinitril, pyrifenox, pyroxychlor, pyroxyfur, pyrimidine fungicides: bupirimate, diflumetorim, dimethirimol, ethirimol, fenarimol, ferimzone, nuarimol, triarimol, anilinopyrimidine fungicides: cyprodinil, mepanipyrim, pyrimethanil, pyrrole fungicides: fenpiclonil, fludioxonil, fluoroimide, quinoline fungicides: ethoxyquin, halacrinate, 8-hydroxyquinoline sulfate, quinacetol, quinoxyfen, tebufloquin, quinone fungicides: benquinox, chloranil, dichlone, dithianon, quinoxaline fungicides: chinomethionat, chlorquinox, thioquinox, thiazole fungicides: ethaboxam, etridiazole, isotianil, metsulfovax, octhilinone thiabendazole, thifluzamide, thiazolidine fungicides: flutianil, thiadifluor, thiocarbamate fungicides: methasulfocarb, prothiocarb, thiophene fungicides: ethaboxam, silthiofam, triazine fungicides: anilazine, triazole fungicides: amisulbrom, bitertanol, fluotrimazole, triazbutil triazolopyrimidine fungicides: ametoctradin, urea fungicides: bentaluron, pencycuron, quinazamid, urea, unclassified fungicides: acibenzolar, acypetacs, allyl alcohol, benzalkonium chloride, benzamacril, bethoxazin, carvone, chloropicrin, DBCP, dehydroacetic acid, diclomezine, diethyl pyrocarbonate, fenaminosulf, fenitropan, fenpropidin, formaldehyde, furfural, hexachlorobutadiene, iodomethane, isoprothiolane, methyl bromide, methyl isothiocyanate, metrafenone, nitrostyrene, nitrothal-isopropyl, OCH, 2-phenylphenol, phthalide, piperalin, proquinazid, pyroquilon, sodium orthophenylphenoxide, spiroxamine, sultropen, thicyofen, tricyclazole, zinc naphthenate.

28. A gelatinous elastomer composition comprising from about 1.0% to about 50.0% by weight of a block copolymer, from about 1% to 99% by weight an oil mixture comprising a mid-block solubilizing oil and a triglyceride, wherein
the triglyceride is present in from about 5% to about 55% percent by weight, based on the weight of the oil mixture.

29. The gelatinous elastomer composition of claim 28, comprising between about 4% and about 25% by weight of the block copolymer, and between about 40% and about 60% by weight of the mid-block solubilizing oil; wherein
the triglyceride is present in from about 7% to about 51% percent by weight, based on the weight of the oil mixture.

30. A method of treating a biological condition or disorder comprising administering to an individual in need of such treatment a pharmaceutical composition comprising the gelatinous elastomer composition of claim 1 comprising an effective amount of a pharmaceutically active agent for treatment of said condition or disorder.

31. The method of claim 30 wherein said pharmaceutical composition is administered topically.

32. The method of claim 30 wherein said biological condition or disorder is keloid scars and the active agent is effective for treatment of keloid scars.

33. The method of claim 30 wherein said biological condition or disorder is scarring and the active agent is effective for treatment of scarring.

34. The method of claim 30 wherein said biological condition or disorder is a fungal infection and the active agent is an anti-fungal agent.

35. The method of claim 30 wherein said biological condition or disorder is a bacterial infection and the active agent is an anti-biotic.

36. The method of claim 30 wherein said biological condition or disorder is eczema and the active agent is effective for treatment of eczema.

37. The method of claim 30 wherein said biological condition or disorder is psoriasis and the active agent is effective for treatment of psoriasis.
